(19) United States
(12) Patent Application Publication
(10) Pub. No.: US 2013/0325000 A1
(43) Pub. Date: Dec. 5, 2013

(54) SYSTEMS FOR TRANSCATHETER ABLATION OF ADVENTITIAL OR PERIVASCULAR TISSUE WHILE PRESERVING MEDIAL AND INTIMAL VASCULAR INTEGRITY THROUGH CONVERGENCE OF ENERGY FROM ONE OR MORE SOURCES, AND METHODS OF MAKING AND USING SAME

(71) Applicant: Mark C. Bates, Encinitas, CA (US)

(72) Inventor: Mark C. Bates, Encinitas, CA (US)

(21) Appl. No.: 13/802,351

(22) Filed: Mar. 13, 2013

Related U.S. Application Data
(60) Provisional application No. 61/654,598, filed on Jun. 1, 2012.

(51) Int. Cl. A61B 18/14

(52) U.S. Cl. CPC A61B 18/1492
USPC 606/41

(57) ABSTRACT

Under one aspect of the present invention, a system for performing renal denervation in a patient having an aorta and a renal artery and a branchpoint therebetween includes a flexible catheter comprising a main section, first and second arms, and a bifurcation between the first and second arms, the main section having a proximal end and a distal end, the distal end configured to be disposed in the aorta, the first arm being coupled to the distal end of the main section and configured to be disposed in the renal artery, the second arm being coupled to the distal end of the main section and configured to be disposed in the aorta, the bifurcation between the first and second arms being configured to engage the branchpoint between the aorta and the renal artery.

### CROSS-REFERENCE TO RELATED APPLICATIONS

This application claims the benefit of priority of U.S. Provisional Application Ser. No. 61/654,598, filed Jun. 1, 2012

### FIELD OF THE INVENTION

This application generally relates to systems for performing controlled depth focal tissue ablation for renal denervation, and methods of making and using the same.

### BACKGROUND OF THE INVENTION

Worldwide prevalence estimates indicate that hypertension may affect as many as 1 billion individuals, and approximately 7.1 million deaths per year may be attributable to hypertension. The World Health Organization reports that suboptimal blood pressure is responsible for 62% of cerebrovascular disease and 49% of ischemic heart disease (IHD) and, as a result, is the number one attributable risk factor for death throughout the world. Unfortunately, some patients continue to have suboptimal control of blood pressure even on maximal medication. The morbidity and mortality risks associated with hypertension support the need for new therapies that can reduce or eliminate the challenges of side effects and poor long-term compliance associated with commercially available blood pressure-lowering medications while at the same time offering hope to those patients with continued hypertension that is resistant to maximal medical therapy

One such new treatment alternative for hypertension, renal denervation, involves disrupting the renal sympathetic nerves, which have long been recognized as playing a significant role in the development of hypertension. As illustrated in FIG. 1A, kidney 110 receives blood via abdominal aorta 130, renal artery 120, and branch vessels 140. Renal sympathetic nerves 150 generally follow abdominal aorta 130 and renal artery 120 allowing for communication between the brain and kidney 110, and include ganglia 160. In the view illustrated in FIG. 1A, renal artery 120 projects somewhat out of the page so as to approximately represent the anterior projection of the renal artery out of the coronal plane of the body. FIG. 1B illustrates a rotated, cross-sectional view of kidney 110, abdominal aorta 130, renal artery 120, branch vessels 140, and renal sympathetic nerves 150, through a plane that bisects aorta 130 and renal artery 120 (defined herein to be the x-y plane). In FIG. 1B, it may be seen that renal sympathetic nerves 150 and ganglia 160 tend to lie primarily along the superior (upper) aspect of branchpoint 170 between aorta 130 and renal artery 120. Additionally, it should be noted that the angle φ between aorta 130 and renal artery 120 may vary from patient to patient. FIG. 1C illustrates a microscope image of a partial cross-section of renal artery 120 through line C-C illustrated in FIG. 1A. As may be seen in FIG. 1C, renal artery 120 includes vessel lumen 130, intima 131, media 132, and adventitia 133, where intima 131, media 132, and adventitia 133 together may be considered to form the “wall” of the renal artery. Renal sympathetic nerves 150 tend to be nestled into and/or disposed immediately outside of adventitia 132.

Referring still to FIGS. 1A-1C, renal denervation procedures attempt to disrupt signaling between the brain and kidney 110, and thus treat or ameliorate hypertension, by damaging renal sympathetic nerves 150. For example, a procedure known as surgical splanchnicecomy was developed in the mid-twentieth century to physically sever renal and peri-aortic sympathetic nerves 150. Although this procedure was often observed to reduce blood pressure, it has fallen out of favor because it is invasive, risks perforation of the wall 131, 132, 133 of renal artery 120, and is associated with unpleasant side effects.

More recently, minimally invasive ablation-based procedures have been developed in which a relatively stiff, steerable ablative tip is percutaneously introduced into renal artery 120 via the peripheral arterial system and abdominal aorta 130. The tip is steered into contact with a portion intima 131 and actuated so as to apply ablation energy, such as radiofrequency (RF) energy, to that portion of intima 131. The ablation energy heats that portion of intima 131, which in turn heats an adjacent portion of media 132, which in turn heats an adjacent portion of adventitia 133, which in turn heats an adjacent portion of one or more renal sympathetic nerves 150. Such heating damages that portion of the nerve(s) 150 and thus disrupts signaling between the brain and kidney 110 along that nerve(s). The ablative tip may be repositioned and the ablation procedure repeated at multiple portions of intima 131, with the goal of damaging a sufficient number of renal sympathetic nerves 150 to sufficiently disrupt signaling between the brain and kidney 110, and thus treat or ameliorate hypertension.

Balloons and helical devices have been used to maintain contact between the energy source and a portion of intima 131, but these devices also rely on propagation of thermal energy from the lumen surface outward. In the case of radiofrequency ablation, ionic conduction and vibration of dipole molecules following alterations of the fields lead to an increase kinetic energy which is converted to heat. This resistive or ohmic heating is greatest at the point of contact. The deeper tissue planes are heated by conduction and, in order to impact the adventitia, transmural injury is needed with the largest amount of tissue damage occurring at intima 131. The injury pattern would take a triangular shape or teardrop shape with the apex being within the targeted adventitia 133 while the most amount of damage is occurring in intima 131 followed by media 132. The therapeutic effect is also negatively impacted by conduction of heat away from the vessel in the adventitia due to flow in the vasa vasorum.

As such, the temperature at intima 131 and 132 may be significantly higher than that at nerves 150, and therefore intima 131 and media 132 are likely to be damaged. While the long-term effects of such damage to intima 131 and media 132 are not yet known, it is believed that such damage potentially may cause blockages in the renal artery, endothelial dysfunction, spasm, dissection, and even perforation or thrombosis, and thus potentially may impair function of kidney 110. The risk of excess energy by utilizing electrical impedance feedback or temperature feedback may decrease the risk of perforation, but such techniques are unlikely to eliminate the risk of thrombus formation and so-called “steam pop” that can be devastating. In addition, it is also not known whether intima 131 grows back after such damage, and if so over what time period, nor whether endothelial cells of intima 131 may function properly even if they do grow back. Moreover, because such a procedure does not provide a means for determining the position of the ablative tip relative to renal sympathetic nerves 150, repeating the ablation procedure at different locations in renal artery 120 with the hopes of sufficiently damaging the nerves in turn may damage as many portions of intima 131 and media 132, thus compounding the aforementioned risks. Additionally, it is known that kidney 110 moves on average about 5 centimeters each time the patient breathes. Such dynamic changes may reduce the likelihood that the ablative tip will remain in contact with a desired portion of intima 131, and furthermore may increase the likelihood that the ablative tip will inadvertently enter one of branch vessels 140, which have relatively thin walls and relatively small diameters. If ablation energy is inadvertently applied within one of branch vessels 140, it potentially may cause immediate and catastrophic perforation and/or intense spasm of that vessel, and potentially also may cause long-term damage to that vessel such as stenosis or psuedoaneurysm. Lastly, because the angle φ between aorta 130 and renal artery 120 may vary significantly from patient to patient, the steering mechanism for the ablative tip may not have a sufficient range of motion to enter the renal artery.

Thus, what is needed is a system and method for more safely and effectively performing renal denervation in a manner compatible with a variety of anatomical variations.

### SUMMARY OF THE INVENTION

Embodiments of the present invention provide systems for performing renal denervation at depths beyond the media of the renal arterial wall by focusing energy from multiple ablation pads, and methods of making and using the same. Such a system may include a flexible catheter with first and second arms each having an ablation pad, the first arm being configured for use in the renal artery and the second arm being configured for use in the abdominal aorta. A bifurcation between the first and second arms is configured to engage the branchpoint between the abdominal aorta and the renal artery. Such engagement between the branchpoint and the bifurcation secures the first arm in the renal artery and the second arm in the aorta, thus reducing the risk of relative motion between the flexible catheter and the kidney as the patient breathes. Moreover, such engagement aligns the ablation pads of the first and second arms relative to each other and to the sympathetic renal nerves such that ablation energy selectively may be applied to a point beyond the media of the renal arterial wall at which location the sympathetic renal nerves are particularly likely to be disposed. The catheter may include one or more articulable joints that facilitate engagement between the branchpoints to compensate for variations in the patient's anatomy and/or that facilitate selection of the particular point to which the ablation energy is provided.

In some embodiments, ablation energy is applied from both of the pads simultaneously, and converges or adds at the desired point. The sum of the energy at the desired point is higher than the energy at either point at which the pads contact the vessel walls, allowing for sufficient energy to damage renal sympathetic nerves at that point e.g., in the adventitia, while preserving the intima and medial vessel structures.

In other embodiments, ablation energy is applied from one pad to the other pad (e.g., in a bipolar arrangement where the polls are separated into each arm), and crosses through the desired point with sufficient energy to damage renal sympathetic nerves at that point. In either embodiment, the risk of damaging the intima and media of the renal arterial wall, as well as the branch vessels off the renal artery, is reduced relative to previously known ablation procedures that use a single ablative tip on a stiff catheter, which may have uncontrolled position relative to the moving kidney and may rely on intense heating of the intima and intermediate tissues to damage the overlying renal sympathetic nerves. Additionally, the present invention is compatible with a variety of different types of ablation energy, and as such the particular construction of the first and second ablation pads may be suitably selected based on the type of ablation energy to be delivered.

Under one aspect of the present invention, a system for performing renal denervation in a patient having an aorta and a renal artery and a branchpoint therebetween includes a flexible catheter comprising a main section, first and second arms, and a bifurcation between the first and second arms, the main section having a proximal end and a distal end, the distal end configured to be disposed in the aorta, the first arm being coupled to the distal end of the main section and configured to be disposed in the renal artery, the second arm being coupled to the distal end of the main section and configured to be disposed in the aorta, the bifurcation between the first and second arms being configured to engage the branchpoint between the aorta and the renal artery.

The system further may include a first ablation pad coupled to the first arm and a second ablation pad coupled to the second arm such that when the bifurcation between the first and second arms engages the branchpoint between the aorta and the renal artery, the first ablation pad engages a wall of the renal artery, and the second ablation pad engages a wall of the aorta. The system further may include first and second ablation energy conductors respectively coupled to the first and second ablation pads, the first and second ablation energy conductors each passing through the main section of the flexible catheter and each being configured to be coupled to an ablation energy source.

In some embodiments, the first and second ablation pads each may be configured to transmit ablation energy from the ablation energy source to a first convergence point beyond the wall of the renal artery and beyond the wall of the aorta, the ablation energy from the first ablation pad and the ablation energy from the second ablation pad constructively adding at the first convergence point, the constructively added energies being sufficient to ablate tissue at the first convergence point. Some embodiments further include a first joint along the main section of the flexible catheter, the first joint being articulable in a first plane. Some embodiments further include a pullwire configured to adjust an angle of the first joint in the first plane so as to configure the first and second ablation pads to transmit ablation energy from the ablation energy source to a second convergence point beyond the wall of the renal artery and beyond the wall of the aorta, the ablation energy from the first ablation pad and the ablation energy from the second ablation pad constructively adding at the second convergence point, the constructively added energies being sufficient to ablate tissue at the second convergence point, for example, at a defined distance from the first convergence point along the outer adventitial area in the renal ostial/aortic branchpoint. Some embodiments include a second joint along the main section of the flexible catheter, the second joint being articulable in a second plane that lies substantially orthogonal to the first plane. The first and second joints each may include a shape memory material.

In other embodiments, the first ablation pad may be configured to transmit ablation energy from the ablation energy source to the second ablation pad (or vice versa), the ablation energy crossing through a point beyond the wall of the renal artery and beyond the wall of the aorta, the ablation energy being sufficient at the point to ablate tissue at the point. The system further may include a first joint along the main section of the flexible catheter, the first joint being articulable in a first plane, and still further may include a pullwire configured to adjust an angle of the first joint in the first plane so as to configure the first ablation pad to transmit ablation energy from the ablation energy source to the second ablation pad through a second convergence point beyond the wall of the renal artery and beyond the wall of the aorta, the ablation energy from the first ablation pad being sufficient to ablate tissue at the second convergence point.

The first and second ablation pads may be configured to emit unipolar radiofrequency (RF) energy, bipolar RF energy, ultrasonic waves, microwave energy, irreversible electroporation, or ionizing radiation. For RF energy emission, the lower the frequency, the deeper the tissue penetration, but also lower resistive heating. In one embodiment, the first ablation pad comprises a bipolar RF electrode configured to contact the aortic wall and the second ablation pad comprises a bipolar radiofrequency electrode configured to contact the renal artery wall. Use of first and second ablation pads permits energy emission at a lower wavelength while permitting adequate tissue penetration depth. The emitted RF energy transacts and has an additive impact at a defined depth which may be the adventitia and specifically the renal sympathetic nerves. It is understood that arterial tissue permittivity is very complex and could be impacted by atherosclerotic changes in the aorta or renal artery so electrical impedance or temperature feedback could be utilized to optimize the treatment. In addition, adding a grounding pad to the patient's skin may have some benefit by optimizing or reducing the human body resonance to more favorable boundary conditions.

In the case of ultrasound, the first and second ablation pads are ultrasound transducers that may be fired simultaneously in the aorta and renal artery so the ultrasonic energy converges and the waves couple where there is a resultant shift in frequency at the point of intersection resulting in an amplified effect at the targeted level compared to the intimal device interface. For microwave energy emission, microwave fields cause the periodic rotation of water molecules sufficient to break hydrogen bonds and this energy is absorbed in the material as heat. In one embodiment, the first and second ablation pads emit microwave energy that may be adjusted to minimize the near field impact by focusing the energy on the adventitia from two separate microwave energy sources based on predicted adventitial and or nerve absorption frequencies.

The system may include one or more sensors configured to sense a parameter, e.g., temperature, bio-impedance, and a programmable controller configured to receive the sensed parameter from the sensor. The programmable controller may direct the first and second ablation pads to emit energy based on the sensed parameter.

In some embodiments, the system further may include a sheath configured to be disposed within the aorta, the flexible catheter being disposed within the sheath, the sheath being retractable relative to the flexible catheter. The system also may include a guidewire configured to be disposed within the renal artery; and a guidewire lumen defined through the flexible catheter and configured to receive the guidewire. The flexible catheter, when disposed in the sheath, may be guidable to a point adjacent the renal artery by pushing the flexible catheter over the guidewire, and may be configured such that retraction of the sheath at the point exposes the first and second arms and the bifurcation therebetween. The flexible catheter also may be configured such that advancement of the flexible catheter after exposing the first and second arms causes the bifurcation between the first and second arms to engage the branchpoint between the renal artery and the aorta. Some embodiments further include a pullwire and a joint along the main section of the flexible catheter, the joint being articulable in a first plane, an angle of the joint being selectable by retracting the pullwire so as to apply additional force in a direction normal to the branchpoint between the renal artery and the aorta.

Under another aspect of the present invention, a method for performing renal denervation in a patient having an aorta and a renal artery and a branchpoint therebetween includes providing a flexible catheter comprising a main section, first and second arms, and a bifurcation between the first and second arms, the main section having a proximal end and a distal end, the distal end configured to be disposed in the aorta, the first and second arms each being coupled to the distal end of the main section; disposing the first arm in the renal artery; disposing the second arm in the aorta; and engaging the branchpoint between the aorta and the renal artery with a bifurcation between the first and second arms.

Methods of making flexible catheters for use in performing renal denervation are also provided.

### BRIEF DESCRIPTION OF DRAWINGS

FIGS. 1A-1B respectively schematically illustrate perspective and cross-sectional views of the kidney, the abdominal aorta, the renal artery, branch vessels, and renal sympathetic nerves.

FIG. 1C is a cross-sectional image of a portion of the renal artery, including the vessel lumen and wall and overlying renal sympathetic nerves.

FIG. 2A schematically illustrates a flexible catheter including multiple ablation pads, according to some embodiments of the present invention.

FIG. 2B schematically illustrates exemplary positioning of the flexible catheter of FIG. 2A in the renal artery and abdominal aorta, according to some embodiments of the present invention.

FIGS. 3A-3I schematically illustrate cross-sections through various points of the flexible catheter of FIG. 2A, according to some embodiments of the present invention.

FIGS. 4A-4B schematically illustrate adaptation of the flexible catheter of FIG. 2A to different anatomical variations of the renal artery, according to some embodiments of the present invention.

FIGS. 5A-5C schematically illustrate articulation of a joint in the flexible catheter of FIG. 2A so as selectively to ablate multiple points outside of the media of the renal arterial wall, according to some embodiments of the present invention.

FIG. 5D schematically illustrates a cross-section of the renal arterial wall following ablation at multiple points outside of the media thereof, according to some embodiments of the present invention.

FIGS. 6A-6C schematically illustrate articulation of a joint in the flexible catheter of FIG. 2A so as selectively to ablate multiple points outside of the media of the renal arterial wall, according to some embodiments of the present invention.

FIG. 7 illustrates steps in a method of forming the flexible catheter of FIG. 2A, according to some embodiments of the present invention.

FIGS. 8A-8B schematically illustrate the flexible catheter of FIG. 2A disposed within a retractable sheath for use during a renal denervation procedure.

FIGS. 9A-9B respectively illustrate steps in alternative methods of disposing the flexible catheter of FIG. 2A within the renal artery and abdominal aorta, according to some embodiments of the present invention.

FIGS. 10A-10B respectively illustrate steps in alternative methods of performing renal denervation using the flexible catheter of FIG. 2A after disposing the catheter within the renal artery and abdominal aorta using the methods of FIGS. 9A or 9B, according to some embodiments of the present invention.

### DETAILED DESCRIPTION

Embodiments of the present invention provide systems for performing renal denervation at depths beyond the media of the renal arterial wall using multiple ablation pads, and methods of making and using the same. The systems include flexible catheters configured to engage the branchpoint between the renal artery and the aorta in such a manner that both inhibits relative motion of the kidney and the catheter as the patient breathes, and facilitates ablation at points that lie outside of the media of the renal arterial wall. Specifically, the flexible catheters include first and second arms that are respectively configured to be disposed in the renal artery and the aorta, with a bifurcation therebetween that engages the branchpoint between the renal artery and the aorta so as essentially to lock the catheter into position with respect to the renal artery and the aorta. A first ablation pad, disposed on the first arm of the catheter, and a second ablation pad, disposed on the second arm of the catheter, may be configured so as to emit ablation energy towards the same point as one another when actuated. As such, the energy emitted by each of the pads may converge and constructively add at that point, and thus generate a higher temperature (or, more generally, a greater amount of ablation energy) at that point than at other points in the surrounding tissue, e.g., than at the intima or media of the renal arterial wall. The point may lie outside of the media, e.g., may lie within the adventitia, or even may lie outside of the adventitia, and preferably lies in a region likely to be occupied by a renal sympathetic nerve. Alternatively, the first ablation pad may be configured to emit ablation energy toward the second ablation pad such that the energy crosses through the desired point. Such an arrangement may generate similar temperatures in tissue that lies along the ablation energy's path between the first and second ablation pads, in contrast to previously known methods that rely on generating relatively higher temperatures at the intima to sufficiently heat the renal sympathetic nerves. The present invention is compatible with a variety of different types of ablation energy, and as such the particular construction of the first and second ablation pads may be suitably selected based on the type of ablation energy to be delivered.

First, an overview of the design of the flexible catheter and its configuration when used during a renal denervation procedure will be provided. Then, further detail will be provided on the construction and control of the flexible catheter. Methods of making the flexible catheter, disposing the catheter into a patient's body, and using the flexible catheter during a renal denervation procedure will be described. Some alternative embodiments will be described throughout, as well as after the discussion of methods of making and using the flexible catheter.

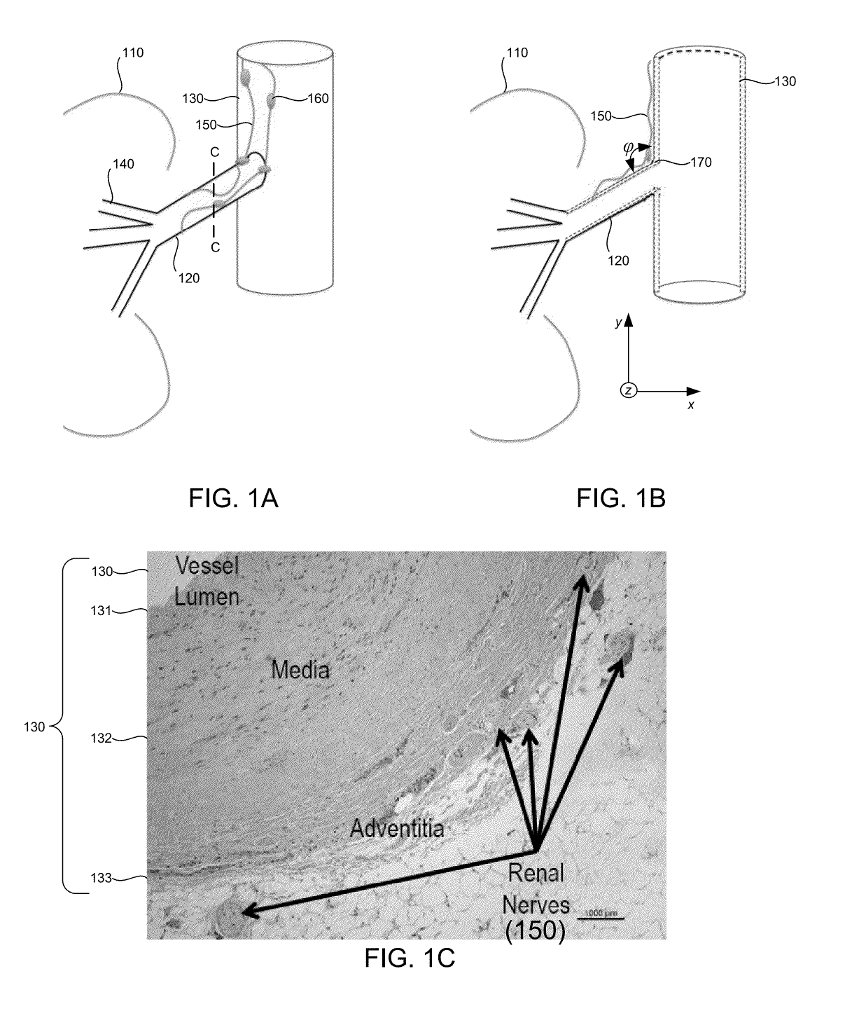

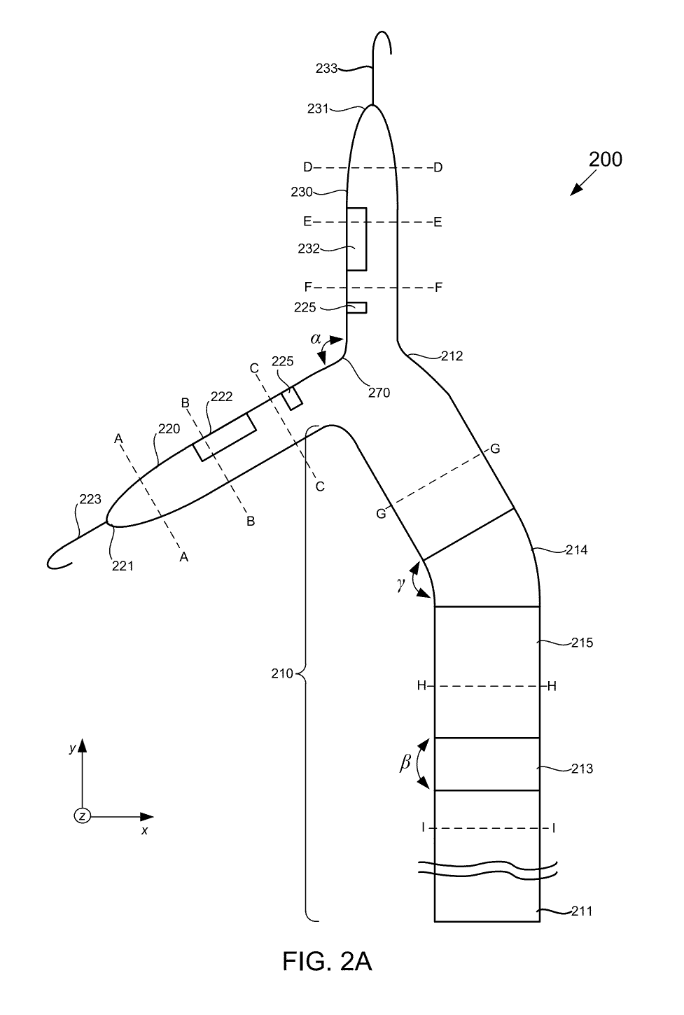

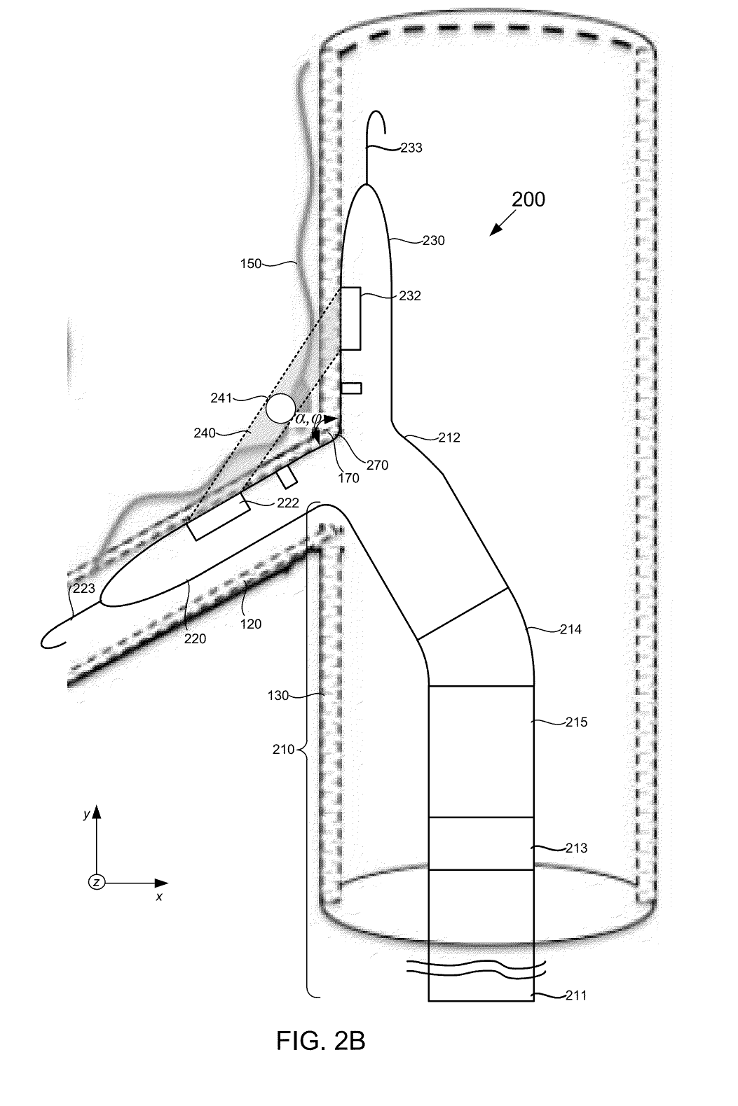

FIG. 2A illustrates flexible catheter 200 constructed according to some embodiments of the present invention. Flexible catheter 200 includes main section 210, first arm 220, and second arm 230, with bifurcation 270 between first arm 220 and second arm 230. Preferably, main section 210, first arm 220, and second arm 230 are formed of a flexible, biocompatible, thermoplastic polymer such as polyethylene, polyurethane, nylon, polyether block amide (PEBAX), or other materials typically used in catheter construction. Preferably, the material from which flexible catheter 200 is formed is sufficiently flexible that first arm 220 is articulable through a wide range of angles α in the x-y plane so as to accommodate a variety of patient anatomies, e.g., variations in the angle φ between aorta 130 and renal artery 120 illustrated in FIG. 1B. Preferably, angle α may range between about 30° and 120°. Although main section 210, first arm 220, and second arm 230 are discussed herein as being separate elements that are coupled to one another, it should be understood that these elements may be different portions of the same unitary structure and may be considered to be coupled to one another via their respective relationships within that structure. Alternatively, one or more of main section 210, first arm 220, and second arm 230 may be separately formed and subsequently coupled to one another.

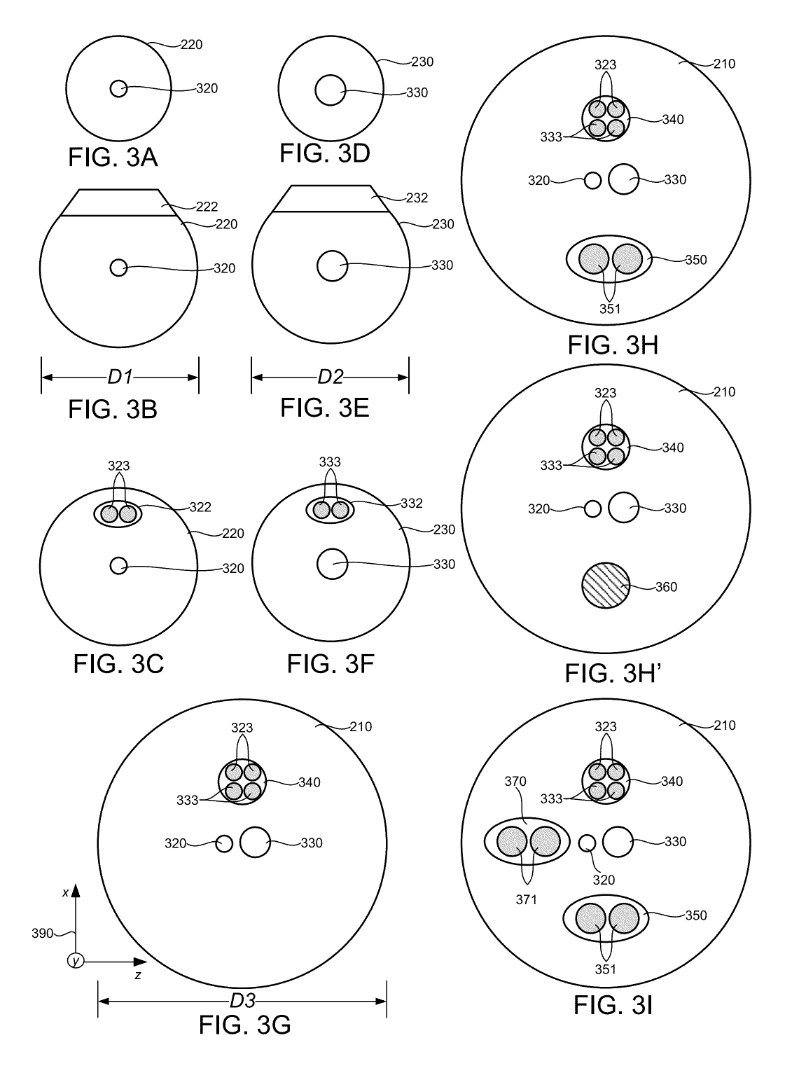

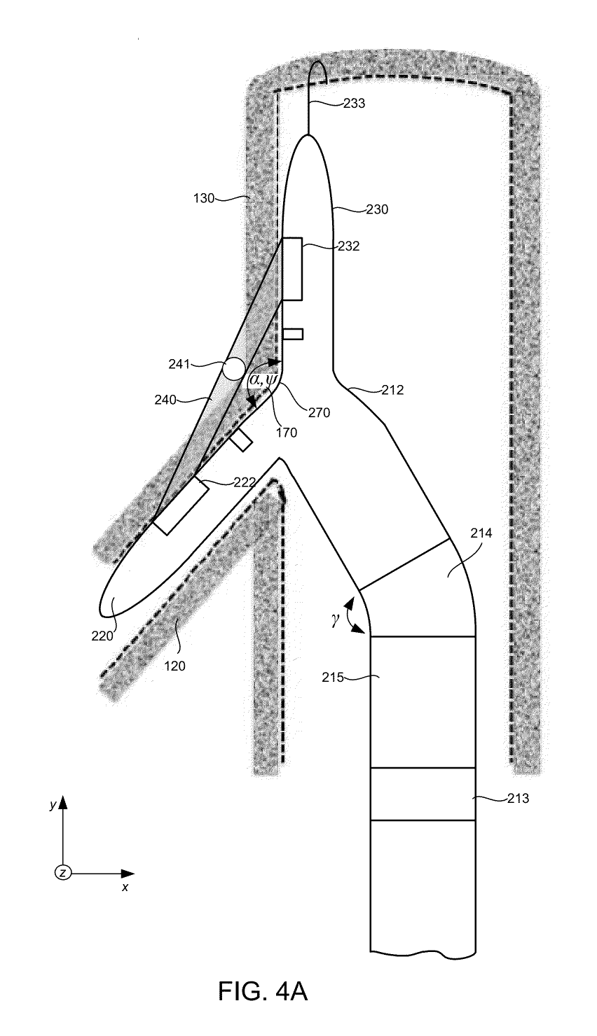

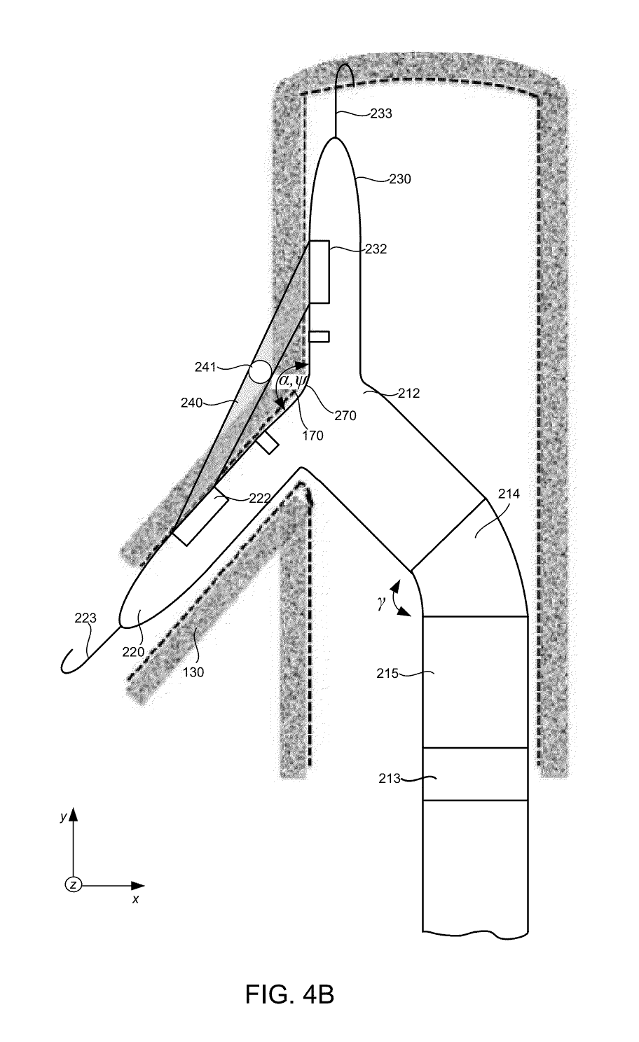

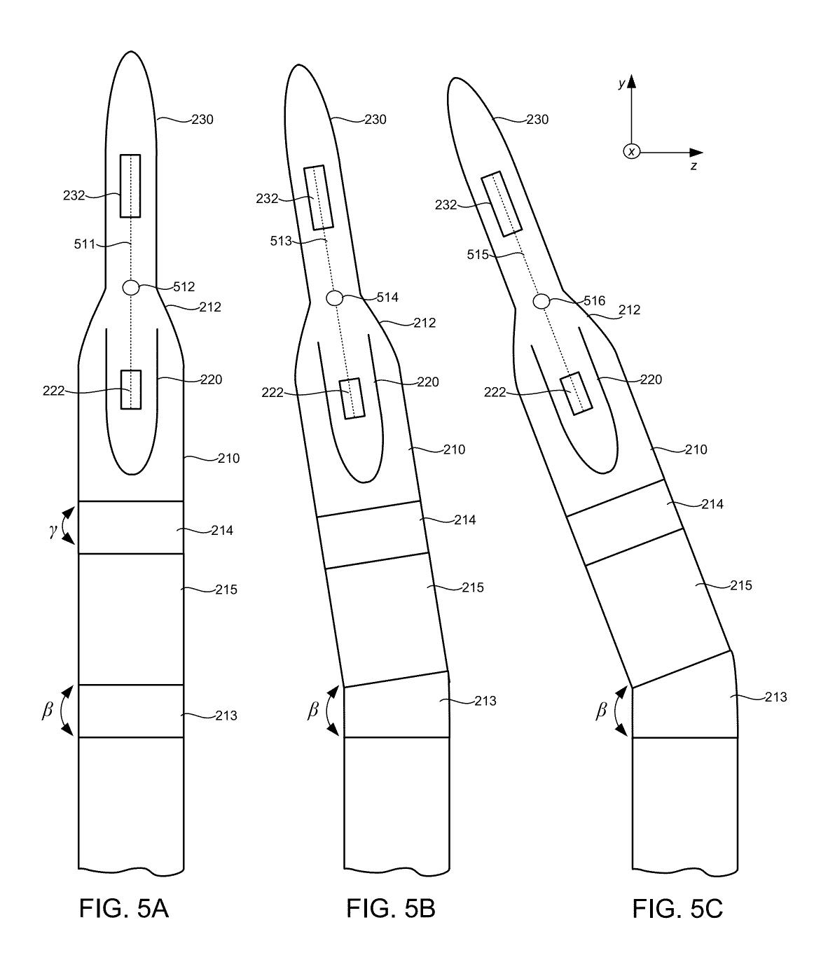

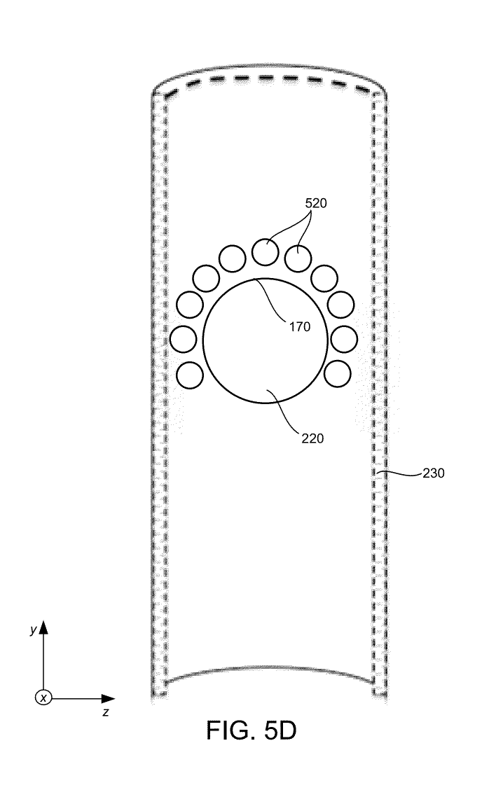

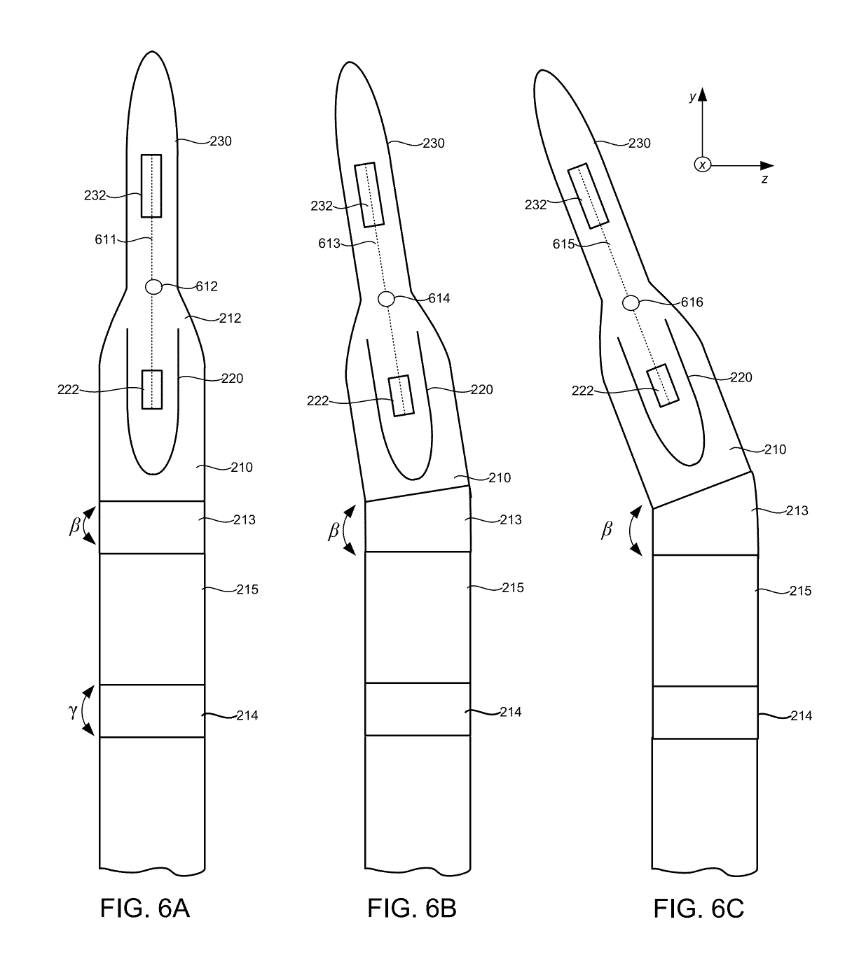

As illustrated in FIG. 2A, main section 210 of flexible catheter 200 includes proximal end 211 configured to lie outside of the patient's body, and distal end 212 configured to be disposed in the patient's abdominal aorta (not shown in FIG. 2A). Main section 210 includes first and second joints 213, 214 that optionally may be separated from one another by intermediate section 215, or alternatively may both be part of the same mechanism as one another. First joint 213 is articulable through angle β in the y-z plane, for example using one or more pullwires as described below with reference to FIGS. 3A-3I, so as to selectively adjust the point to be ablated using flexible catheter 200 as described below with reference to FIGS. 5A-6C. Preferably, angle β has a nominal (or default) value of 0° and may range between about +90° (orthogonal to plane of paper and in +z direction) and an angle of about −90° (orthogonal to plane of paper and in −z direction. Second joint 214 is articulable through angle γ in the x-y plane, which is orthogonal to the y-z plane, for example using a resilient shape-memory material or using one or more pullwires as described below with reference to FIGS. 3A-3I, so as to compensate for variations in the patient's anatomy and to enhance engagement between bifurcation 270 of flexible catheter 200 and branchpoint 170 between aorta 130 and renal artery 120 (not shown in FIG. 2A), as described in greater detail below with reference to FIGS. 4A-4B. Preferably, angle γ has a nominal (default) value of about 110°, and may range between about 90° and about 120° in the x-y plane. So as to facilitate their articulation through the desired angles, first and second joints 213, 214 each may include a pleated, accordion-like structure that facilitates bending of the joints through a suitable range of angles.

Preferably, main section 210 has a length suitable for distal end 212 to reach branchpoint 170 via the peripheral arterial system and abdominal aorta 130, while proximal end 211 remains outside of the patient's body, and a diameter suitable for use within the peripheral arterial system and abdominal aorta. In one illustrative embodiment, main section 210 has a diameter e.g., a diameter of about 3 mm or less, suitable for use with a sheath having a 9 French or smaller diameter.

First arm 220 of flexible catheter 200 is coupled to distal end 212 of main section 210, and is configured to be disposed in renal artery 120 (not shown in FIG. 2A). First arm 220 includes tapered distal tip 221 and first ablation pad 222. Preferably, first arm 220 has a length and a diameter suitable for use within the renal arteries of patients with a wide variety of possible anatomies. For example, first arm 220 may have a length of about 1.5 inches (about 38 mm) between bifurcation 270 and distal tip 221, so as to be usable in any patients having renal arteries with lengths greater than or equal to about 1.5 inches (about 38 mm), a length of about 2.5 inches (about 63.5 mm) being the mean renal artery length. First arm 220 may have a diameter of about 1.5 mm, so as to be usable in patients having renal arteries with diameters greater than about 1.5 mm, a diameter of about 6 mm being the mean renal artery diameter.

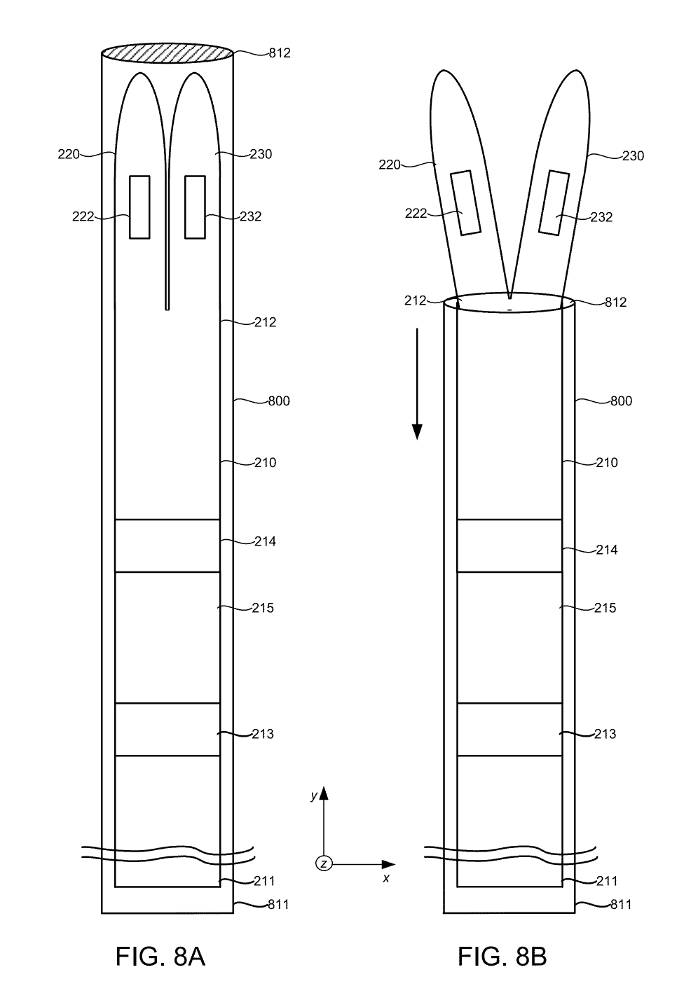

Second arm 230 of flexible catheter 200 also is coupled to distal end 212 of main section 210, and is configured to be disposed in abdominal aorta 130 (not shown in FIG. 2A). Second arm 230 includes tapered distal tip 231 and second ablation pad 232. Preferably, second arm 230 has a length and a diameter suitable for use within the aortas of patients with a wide variety of possible anatomies. For example, second arm 230 may have approximately the same dimensions as first arm 220, or even may have larger dimensions than first arm 220, because the abdominal aorta is significantly longer and wider than the renal artery. Preferably, first and second arms 220, 230 together have approximately the same diameter as does main section 210, so that all three of these components of flexible catheter 200 readily may be disposed within a retractable sheath that may be used to introduce the flexible catheter into the aorta and renal artery, such as described in greater detail below. In one illustrative embodiment, first and second arms 220, 230 each have a diameter of about 1.5 mm and main section 210 has a diameter of about 3.0 mm, so that flexible catheter 200 readily may be disposed inside of a 9 French or smaller sheath in the manner described below with reference to FIGS. 8A-8B.

First arm 220 may be configured to receive renal guidewire 223 therethrough to facilitate positioning of first arm 220 within renal artery 120, and second arm 230 may be configured to receive aorta guidewire 233 therethrough to facilitate positioning of second arm 230 within aorta 130, such as described in greater detail below. In an alternative embodiment, aorta guidewire 233 may be omitted, and the shape of catheter 200 instead may cause second arm 230 to be guided into aorta 130 as first arm 220 is guided over renal guidewire 223 into renal artery 120. The distal ends of guidewires 223 and/or 233 may be atraumatically shaped (e.g., using a “J” shape such as illustrated in FIG. 2A) so as respectively to reduce the likelihood of damaging renal artery 120 and aorta 130.

Sensors 225 may be disposed on main section 210, first arm 220, second arm 230, or any combination thereof. Sensors 225 are configured to sense a parameter, e.g., temperature, bio-impedance, and to send a signal to a programmable controller, described below, based on the sensed parameter.

FIG. 2B illustrates flexible catheter 200 positioned within abdominal aorta 130 and renal artery 120 and securely engaged with branchpoint 170 between the aorta and the renal artery. Flexible catheter 200 is generally disposed in a plane that bisects aorta 130 and renal artery 120 (defined herein to be the x-y plane). First arm 220 of flexible catheter 200 is disposed within renal artery 120, second arm 230 of flexible catheter 200 is disposed within aorta 130, and main portion 210 of flexible catheter 200 is also disposed within aorta 130. Bifurcation 270 between first arm 220 and second arm 230 is securely engaged with branchpoint 170 between aorta 130 and renal artery 120. Additionally, angle α between first arm 220 and second arm 230 is substantially equal to angle φ between aorta 130 and renal artery 120. As such, flexible catheter 200 substantially conforms to the walls of aorta 130 and renal artery 120 in the regions adjacent branchpoint 170, which places first ablation pad 222 into contact with wall 131, 132, 133 of renal artery 120, and places second ablation pad 232 into contact with the wall of aorta 130.

As illustrated in FIG. 2B, ablation energy generated by first ablation pad 222 and/or second ablation pad 232, e.g., generated by one or more ablation energy generators to which ablation pads 222, 232 are coupled via suitable conductors such as described in greater detail below, traverses path 240 between the ablation pads. In particular, the ablation energy crosses through point 241, which lies adjacent to branch point 170 and through which it is likely that renal sympathetic nerves 150 pass. The particular type and intensity of the ablation energy, and the manner in which first and second ablation pads 122 transmit the energy into point 241, is selected so as to reduce the heating of the walls of renal artery 120 and aorta 130, and to increase the convergent ablation energy at point 241 so as to increase the likelihood of damaging any renal sympathetic nerves passing therethrough, relative to what could be achieved with a single ablative pad or ablative tip.

For example, in some embodiments, first and second ablation pads 222, 232 each respectively may emit similar amounts and types of ablative energy as one another toward point 241. The energy from pad 222 may traverse path 240 towards point 241, and the energy from pad 232 similarly may traverse path 240 towards point 241. The energy from pads 222, 232 reach point 241 at substantially the same time as one another point 241 and may constructively add with one another, causing point 241 to receive about twice as much energy as it would from only a single one of pads 222, 232, and ablating the tissue (including any renal sympathetic nerves 150) present at point 241. Because the energy from the two pads constructively adds at point 241, the amount of ablative energy at point 241 may be significantly higher than would be achievable using only a single ablative pad or tip emitting that amount and type of ablative energy, so comparatively less energy applied to the walls of renal artery 120 and aorta 130 may be required to achieve similar damage to nerves 150.

It should be recognized that pads 222, 232 need not necessarily emit the same amount of ablative energy as one another to achieve such an effect, nor need they emit that energy at substantially the same time as one another. For example, pads 222, 232 need not necessarily be equidistant from bifurcation 270. If pads 222, 232 are different distances from bifurcation 270 than one another, then the amount of time required for ablative energy to travel from pad 222 to point 241 may be different than the amount of time required for ablative energy to travel from pad 232 to point 241, and the amount of energy received at point 241 from pads 222 and 232 similarly may differ because of the differing volumes and/or types of tissue with which the energy interacts along path 240 before reaching point 241. The respective amount of energy and the timing of the emission of such energy from pads 222 and 232 suitably may be adjusted so as to reach any desired point 241. Moreover, the relative phases of the ablative energy emitted from pads 222 and 232 suitably may be adjusted so as to enhance the constructive addition of the energy at point 241.

Additionally, it should be recognized that the energy emitted by pads 222, 232 need not necessarily exclusively traverse path 240. To the contrary, it is likely that the energy emitted by pad 222 may spread out as it travels away from pad 222, and similarly the energy emitted by pad 232 may spread out as it travels away from pad 232. So long as a sufficient amount of energy from pads 222, 232 converges and constructively adds at point 241, tissue at that point may be sufficiently ablated.

In other embodiments, first and second ablation pads 222, 232 are used in a bipolar arrangement, e.g., in which pad 222 is placed at a high potential and pad 232 is placed at a low potential, and current is driven from pad 222 to pad 232 along path 240 and through path 241 (or vice versa). Such an arrangement may reduce the heating of the walls of the renal artery 120 and aorta 130 relative to that which may be caused by a single one of pads 222 or 232 in a monopolar arrangement emitting an otherwise similar amount of energy, because the tissue along path 240 (including point 241) may be relatively evenly heated. By comparison, using a single ablative pad 222 requires heating the renal artery or aorta wall to a relatively high temperature to achieve a somewhat lower, but still sufficient, temperature at renal sympathetic nerve 150. As discussed above for the embodiment in which both pads emit energy towards point 241, in the bipolar arrangement pads 222, 232 need not be equidistant from point 241.

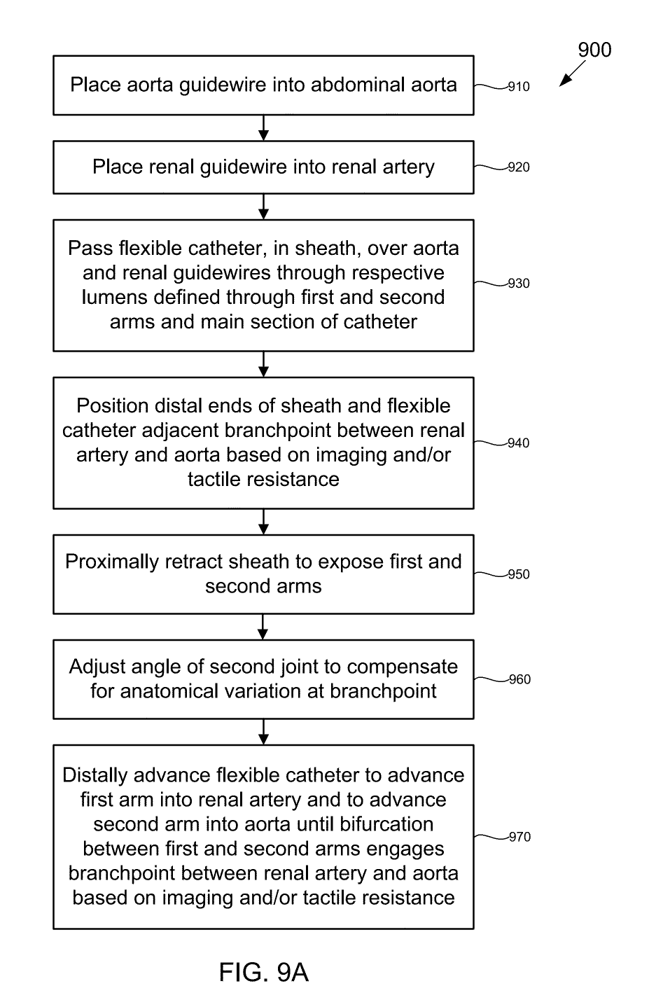

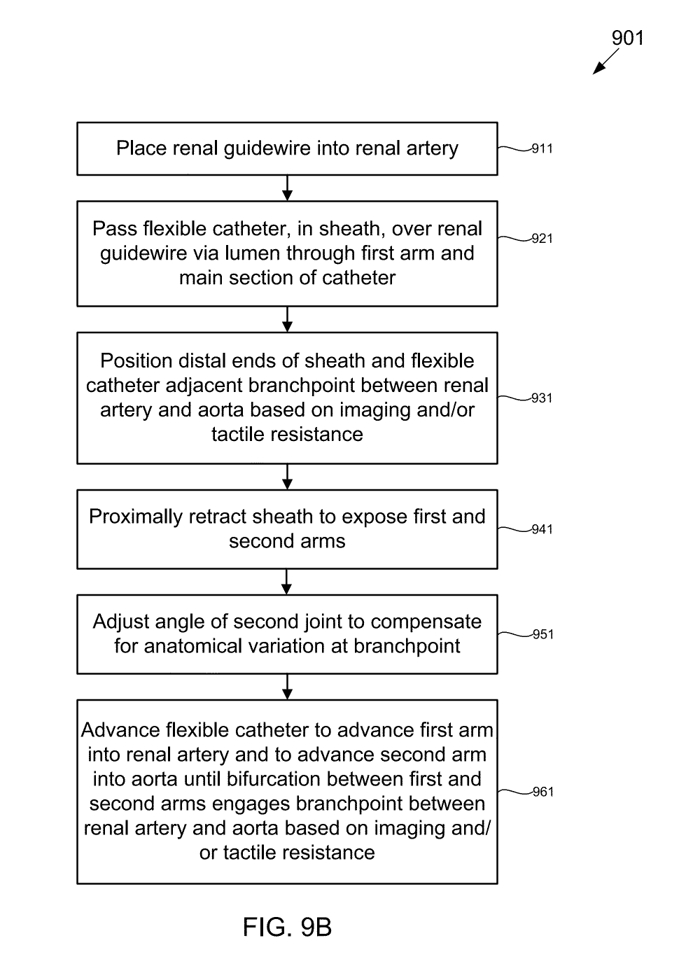

In still other embodiments, first and second ablation pads 222, 232 may be actuated at different times than one another. For example, pad 222 may be actuated to emit ablation energy along path 240 and through point 241 at a first time, and pad 232 may be actuated to emit ablation energy along path 240 and through point 241 at a second time that is later than the first time. Preferably, the total amount of energy received at point 241 is sufficiently high to damage the tissue at that point, while the total amount of energy respectively received by the walls of renal artery 120 and aorta 130 is sufficiently low to inhibit significant damage to those walls. In some such embodiments, first and/or second ablation pads 222, 232 may be configured to selectively focus ablation energy at point 241 so as to increase the relative amount of tissue damage at that point. Moreover, note that in some such embodiments, only one of first and second ablation pads 222, 232 need be used to achieve such tissue damage; indeed, the other of first and second ablation pads 222, 232 optionally may be omitted from catheter 200. Even if only one of first and second ablation pads 222, 232 is included (or used) in catheter 200, the overall configuration of catheter 200 still may beneficially provide ease of deployment and engagement with renal artery 120 and aorta 130 in the manner described below with reference to FIGS. 9A-9B.

Turning now to FIGS. 3A-6C, the construction and control of flexible catheter 200 will be further described.

FIGS. 3A-3I respectively illustrate cross-sections through various portions of flexible catheter 200 as designated by lines A-A through I-I in FIG. 2A. Specifically, FIG. 3A illustrates a cross-section along line A-A through tapered tip 221 of first arm 220 of catheter 200. Lumen 320 is defined through first arm 220, and is configured to receive renal guidewire 223 so as to aid in guiding first arm 220 into renal artery 120 during preparation for a renal denervation procedure, such as described further below with reference to FIGS. 9A-9B. Similarly, FIG. 3D illustrates a cross-section along line D-D through tapered tip 231 of second arm 230 of catheter 200. Lumen 330 is defined through second arm 230, and configured to receive aorta guidewire 233 so as to aid in guiding second arm 230 into aorta 130 during preparation for a renal denervation procedure, such as described further below with reference to FIGS. 9A-9B. Note that lumens 320 and 330 need not necessarily be the same size as one another. For example, renal guidewire 223 may have a diameter of 0.14 inches, with lumen 320 being suitably slightly larger than guidewire 223, while aorta guidewire 233 may have a diameter of 0.35 inches, with lumen 330 being suitably slightly larger than guidewire 233.

FIG. 3B illustrates a cross-section along line B-B through first arm 220 and first ablative pad 222 of catheter 200. In this cross-section, first arm 220 may be seen again to have lumen 320 defined therethrough, as well as to include ablative pad 222 coupled thereto. First arm 220 has an outer diameter D1, which as noted above may be selected to be compatible with a wide range of possible patient anatomies of renal artery 120, as well as with percutaneous placement therein via a 9 French or smaller delivery sheath or guide catheter. In one example, diameter D1 is about 1.5 mm or less. Similarly, FIG. 3E illustrates a cross-section along line E-E through second arm 230 and second ablative pad 232 of catheter 200. In this cross-section, second arm 230 has an outer diameter D2, which as noted above may be selected to be compatible with a wide range of possible patient anatomies of aorta 130, as well as with percutaneous placement therein via a 9 French or smaller delivery sheath. In one example, diameter D2 is about 1.5 mm or less, noting that D2 suitably may be the same or different than D1. Second arm 230 may be seen again to have lumen 330 defined therethrough, as well as to include ablative pad 232 coupled thereto.

Ablative pads 222, 232 illustrated in FIGS. 3B and 3E may have similar constructions as one another, although in some embodiments they may have different constructions than one another. As noted above, the present invention is compatible with a variety of different types of ablation energy, and as such ablative pads 222, 232 may have any suitable construction for emitting a desired amount and type of ablation energy. For example, in some embodiments pads 222, 232 are conductive (e.g., metallic) electrodes configured to emit and/or receive RF energy generated by a separate RF energy generator and coupled thereto by suitable conductors (described further below with reference to FIGS. 3C and 3F). In other embodiments, pads 222, 232 are ultrasonic transducers configured to generate high intensity ultrasonic waves responsive to electrical energy generated by a separate electrical generator coupled thereto by suitable conductors. In still other embodiments, pads 222, 232 are microwave surgical antennas configured to emit microwave energy generated by a separate microwave energy generator and coupled thereto by suitable conductors. Note that pads 222, 232 need not necessarily be constructed to emit ablation energy that heats tissue, and that instead they may be constructed to emit cryo-ablation energy, damaging radiation, and the like. Preferably, first and second ablative pads 222, 232 are constructed so as not to significantly increase the respective outer diameters Da and Db of first and second arms 220, 230, and also such that the pads respectively come into sufficient contact with the walls of renal artery 120 and aorta 130 to deliver ablative energy to the tissue.

FIG. 3C illustrates a cross-section along line C-C through first arm 220 at a point proximal of ablative pad 222. In this cross-section, first arm 220 may be seen again to include lumen 320 defined therethrough, as well as to include conductor lumen 322 through which one or more conductors 323 pass, and to have an outer diameter approximately equal to Dl. Conductor(s) 323 are coupled to ablative pad 222, pass out of the body via main section 210 of catheter 200, and are configured to be coupled to a suitable ablation energy source (pad 222, main section 210, and ablation energy source not illustrated in FIG. 3C). Similarly, FIG. 3F illustrates a cross-section along line F-F through second arm 230 at a point proximal of ablative pad 232. In this cross-section, second arm 230 may be seen again to include lumen 330 defined therethrough, as well as to include conductor lumen 332 through which one or more conductors 333 pass, and to have an outer diameter approximately equal to D2. Conductor(s) 333 are coupled to ablative pad 232, pass out of the body via main section 210 of catheter 200, and are configured to be coupled to a suitable ablation energy source, which may be the same as the ablation energy source to which conductors 323 may be coupled (pad 232, main section 210, and ablation energy source not illustrated in FIG. 3F).

The ablation energy source may be configured to generate any suitable type of ablation energy for use with the particular patient and configuration of catheter 200. Examples of suitable ablation energy generators include RF generators such as the BARD® RF Cardiac Ablation Generator (C.R. Bard Electrophysiology Division, Lowell, Mass.), the GENIUS™ Multi-Channel RF Generator (Medtronic Ablation Frontiers LLC, Carlsbad, Calif.), or the Stockert 70 RF Generator (Biosense Webster, Inc., Diamond Bar, Calif.); microwave generators such as the EVIDENT™ MW Ablation System (Covidien, Mansfield, Mass.) or the MICROTHERMX® Microwave Ablation System (BSD Medical, Salt Lake City, Utah); and ultrasonic generators such as the Integra CUSA NXT™ Ultrasonic Tissue Ablation System or the Integra CUSA EXCEL®+ Ultrasonic Tissue Ablation System (Integra Lifesciences Corporation, Plainsboro, New Jersey). First and second ablation pads 222, 232 suitably are configured to emit ablation energy based on the output of the ablation energy generator coupled thereto respectively via conductors 322, 323. Note that first and second ablation pads 222, 232 may be coupled to the same ablation energy generator as one another, or even to different ablation energy generators than one another.

Energy ablation source may include or may be a coupled to a programmable controller. The programmable controller may comprise a commercially available microcontroller unit including a programmable microprocessor, volatile memory, nonvolatile memory such as EEPROM for storing programming, and nonvolatile storage. The programmable controller may be configured to direct ablation pads 222, 232 to transmit ablation energy at energy levels stored within the programming. In one embodiment, the programmable controller is operatively coupled to sensors 225 disposed on main section 210, first arm 220, and/or second arm 230. In such an embodiment, the programmable controller may be configured to direct ablation pads 222, 232 to transmit ablation energy at a first energy level, monitor sensed parameters, e.g., temperature, bio impedance, from sensors 225, and direct ablation pads 222, 232 to transmit ablation energy at a second energy level based on the sensed parameters. Thereafter, the programmable controller may continue to monitor sensed parameters and continue to adjust ablation energy levels based on the sensed parameters. The programmable controller further may define therapeutic endpoints for ablation energy based on the sensed parameters.

FIG. 3G illustrates a cross-section along line G-G through main section 210 of catheter 200 illustrated in FIG. 2A. In this cross-section, main section 210 may be seen to have defined therein conductor lumen 340 through which conductors 323 and 333 respectively from first and second arms 220, 230 pass. Main section 210 also has defined therein continuations of lumens 320 and 330 respectively from first and second arms 220, 230, which respectively are configured to receive renal guidewire 223 and aorta guidewire 233. Main section 210 has an outer diameter D3 that may be selected to be compatible with a wide range of possible patient anatomies of aorta 130 and with percutaneous placement therein via a 9 French or smaller delivery sheath, as well as to accommodate the various control elements, conductors, and lumens passing therethrough. In some embodiments, D3 is approximately equal to D1+D2, and in one illustrative embodiment D3 is approximately 3 mm. FIG. 3G also includes reference axis 390 illustrating that the cross-section along line G-G lies in the x-z plane, in which plane the cross-sections illustrated in FIGS. 3D-3I all substantially lie because second arm 230 and main section 210 both extend substantially parallel to aorta 230; by comparison, the cross-sections illustrated in FIGS. 3A-3C lie in a different plane (axis not illustrated) defined by the angle α at which first arm 220 relative to aorta 230.

FIG. 3H illustrates a cross-section along line H-H through main section 210. In this cross-section, main section 210 may be seen again to include conductor lumen 340, conductors 323, 333 passing therethrough, and lumens 320, 330, as well as first pullwire lumen 350 having one or more pullwires 351 passing therethrough. Pullwire(s) 351 are coupled to second joint 214, and facilitate articulation of joint 214 through a variety of angles γ in the x-y plane as illustrated in FIG. 2A. The use of such pullwire(s) 351 to adjust angle γ so as to accommodate different possible patient anatomies as described further below with reference to FIGS. 4A-4B.

In an alternative embodiment illustrated in FIG. 3H′, the cross-section along line H-H through main section 210 instead may include reinforcing member 360 in place of pullwire lumen 350 and pullwire(s) 351. Reinforcing member 360 may include a resilient shape memory material configured to maintain second joint 214 at or about a desired angle γ so as to increase the amount of force that catheter 200 exerts in the −x direction, and thus facilitate engagement between bifurcation 270 between first and second arms 220, 230 and branchpoint 170 between renal artery 120 and aorta 130. Preferably, reinforcing member 360 may transition between a relatively straight configuration when catheter 200 is disposed within a delivery sheath, as described further below with reference to FIG. 8A, and a bent configuration maintaining second joint 214 at or about angle γ when the delivery sheath is retracted so that catheter 200 may be used in a renal denervation procedure. In the embodiment illustrated in FIG. 3H′, reinforcing member 360 may extend upwards through joint 214 and into the cross-section illustrated in FIG. 3G, although member 360 is not illustrated in FIG. 3G. Additionally, it should be understood that a combination of reinforcing member 360 and pullwires 351 respectively may be used to maintain second joint 214 about a nominal angle γ and to adjust that angle as appropriate.

Turning again to the primary embodiment, FIG. 31 illustrates a cross-section along line I-I through main section 210. In this cross-section, main section 210 may be seen again to include conductor lumen 340, conductors 323, 333 passing therethrough, lumens 320, 330, and first pullwire lumen 350 having one or more pullwires 351 passing therethrough, as well as second pullwire lumen 370 having one or more pullwires 371 passing therethrough. Pullwire(s) 371 are coupled to first joint 213, and facilitate articulation of joint 213 through a variety of angles β in the x-z plane as illustrated in FIG. 2A. The use of such pullwire(s) 371 to adjust angle β so as to adjust the location of the particular point 241 to be ablated is described further below with reference to FIGS. 5A-6C.

As mentioned above, second joint 214 of main section 210 of catheter 200 may be utilized to accommodate a variety of possible patient anatomies. Specifically, as illustrated in FIG. 4A, in some patients the renal artery 120 may branch off from aorta 130 at a relatively steep angle, designated angle ψ. Indeed, in some patients angle ψ may approach 180°, with renal artery 120 being nearly vertical relative to aorta 130. While catheter 200 is sufficiently flexible that first arm 220 may readily bend in the x-y plane to provide an angle α substantially equal to ψ, the amount of force in the −x direction that bifurcation 270 between first arm 220 and second arm 230 may exert upon branchpoint 170 between renal artery 120 and aorta 130 may be reduced somewhat as a result of this steeper angle, resulting in a relatively reduced degree of engagement between branchpoint 170 and bifurcation 270. To increase the amount of force in the −x direction that bifurcation 270 between first arm 220 and second arm 230 may exert upon branchpoint 170 between renal artery 120 and aorta 130, angle γ of second joint 214 may be decreased, as illustrated in FIG. 4B. Such an adjustment of angle γ may be achieved, for example, using pullwire(s) 351 described above with reference to FIG. 3H, or using reinforcing member 360 described above with reference to FIG. 3H′, or a combination of the two.

While angle γ defined by second joint 214 of flexible catheter 200 may be adjusted to compensate for a variety of possible patient anatomies, angle β defined by first joint 213 may be adjusted to select the particular point 241 to be ablated by using pullwire(s) 371 described above with reference to FIG. 31. For example, as illustrated in FIG. 5A, angle β initially may begin at approximately 0°, corresponding to a straight configuration of main section 210, and ablation performed along a path defined by first line 511 and crossing through first convergence point 512. Then, using pullwire(s) 371, angle β may be adjusted the y-z plane, and ablation performed along a path defined by second line 513 and crossing through second convergence point 514 that is offset in the −y and −z directions from first convergence point 512. Then, using pullwire(s) 371, angle β again may be adjusted the y-z plane, and ablation performed along a path defined by third line 515 and crossing through third convergence point 516 that is offset in the −y and +z directions from second convergence point 514. It should be understood that pullwire(s) 371 also may be used to adjust angle β in the direction opposite from that illustrated in FIGS. 5A-5C, so as to define paths crossing through points that respectively are offset in the −y and +z directions from first convergence point 512.

Because first arm 220 is disposed within renal artery 120, varying angle β causes first arm 220 to rotate along an axis defined by renal artery 120, while the comparatively larger size of aorta 130 allows second arm 230 to be laterally translated. Such a procedure may be repeated at any desired step size and numbers of different angles β permitted by the range of motion of first joint 213 and by the patient's anatomy. For example, FIG. 5D illustrates a cross-section of aorta 230 in the y-z plane, with renal artery 220 projecting in the −x direction, following ablations performed at eleven different angles β using pullwire(s) 371 to adjust first joint 213 (pullwires and joint not illustrated in FIG. 5D). For each corresponding angle β, a different point 520 adjacent to branchpoint 170 between renal artery 220 and aorta 230 is ablated. As such, any renal sympathetic nerves 150 that may pass through points 520 may be ablated, facilitating the treatment of hypertension in the patient.

Note that the relative positions of first and second joints 213, 214 suitably may be selected to modify the particular manner in which the configuration of flexible catheter 200 may be adjusted in situ. For example, in the alternative embodiment illustrated in FIGS. 6A-6C, first joint 213 may be disposed distally of second joint 214, instead of proximally as is illustrated in FIGS. 5A-5C. In a manner similar to that described above with reference to FIGS. 5A-5C, adjusting angle β via pullwire(s) 371 respectively modifies the lines 611, 613, 615 that define the ablation energy path, and thus modifies the positions in the y-z plane of points 612, 614, 616 through which the ablation energy crosses. However, the embodiment illustrated in FIGS. 6A-6C causes somewhat less lateral motion in the z direction of first and second arms 220, 230, while still allowing a variety of points to be ablated in a manner analogous to that illustrated in FIG. 5D. Additionally, joints 213 and 214 may be provided in a single mechanism located at any desired location along main portion 210 of catheter 200.

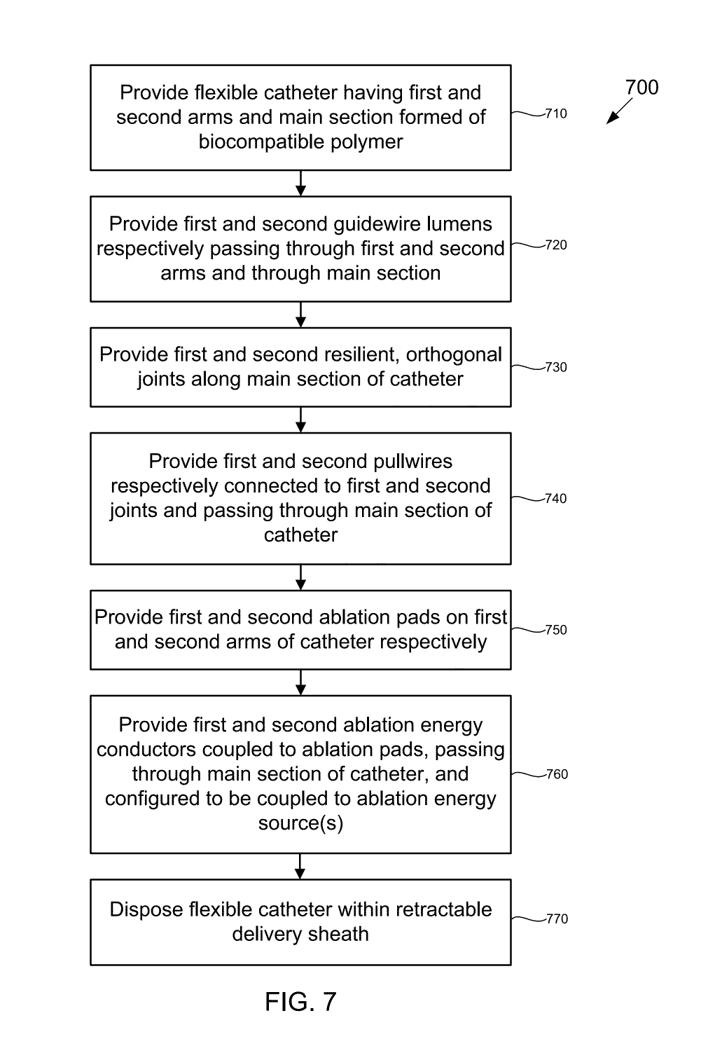

Exemplary methods of making, delivering, and using flexible catheter 200 to perform renal denervation will now be described with reference to FIGS. 7-10B.

Specifically, FIG. 7 illustrates steps in a method 700 of making flexible catheter 200 illustrated in FIG. 2A. Method 700 includes providing a flexible catheter having first and second arms 220, 230 and a main section 210 formed of a biocompatible polymer (step 710). Preferably, first and second arms 220, 230 and main section 210 are of unitary construction with one another, and may be formed, for example, by suitably extruding, blow-molding, or die-casting a flexible, biocompatible, thermoplastic polymer such as polyethylene, polyurethane, nylon, polyether block amide (PEBAX), or other materials typically used in catheter construction. Alternatively, one or more of these components may be formed individually and coupled to the other(s) using suitable bonding, stitching, adhesive, or the like. First and second arms 220, 230 and main section 210 may be formed so as to have nominal (default) angles relative to one another, e.g., angles compatible with a wide range of patient anatomies. For example, first and second arms 220, 230 may be formed with angle α therebetween that is selected to be approximately equal to the mean angle φ/ψ between renal artery 120 and aorta 130 for the anticipated population of patients, e.g., 110°.

First guidewire lumen 320 that passes through first arm 220 and main section 210, and second guidewire lumen 330 that passes through second arm 230 and main section 210, are then provided (step 720). Note that lumens 320, 330 may be defined at the same time as first arm 220, second arm 230, and/or main section 210, or alternatively may be defined at a later time.

First and second resilient joints 213, 214 that are articulable in planes orthogonal to one another then are provided along main section 210 (step 730). In some embodiments, one or both of joints 213, 214 each may include a flexible material, such as a polymer or a shape memory alloy, that holds the joint at a nominal angle, e.g., at an angle of 180° (straight) for joint 213 and an angle of 110° for joint 214, and yet allows the joint to suitably flex. For example, as described above with reference to FIG. 3H′, joint 214 may include resilient member 360 that retains the joint at a nominal angle, and yet allows joint 214 to flex slightly. Joint 213 may include an analogous resilient member. In other embodiments, main section 210 may be formed so as to place one or both of joints 213, 214 at its respective nominal angle. Any resilient members provided in joints 213, 214 may be provided during step 710, e.g., molded directly into main section 210, or alternatively may be inserted at a later time. Optionally, so as to facilitate the articulation of joints 213, 214 through the desired angles, portions of main section 210 adjacent joints 213, 214 may include pleats or, accordion-like folds, which may be defined during step 710 described above.

First and second pullwires 351, 371, respectively connected to first and second joints 213, 214 and respectively disposed within pullwire lumens 350, 370 defined through main section 210 then are provided (step 740). Preferably, first and second pullwires 351, 371 pass out of the body and are controllable by a physician during the renal denervation procedure so as better to conform flexible catheter 200 to the patient's particular anatomy and to select different points to be ablated, as appropriate. Each of pullwires 351, 371 may include one, two or more individual pullwires suitably configured to adjust the angles of joints 213, 214 to a desired degree. As noted above, in some embodiments only one of joints 213, 214 may be controlled via pullwire, in which case the other pullwire may be omitted. Pullwire lumens 350, 370 may be defined during step 720, e.g., at about the same time as guidewire lumens 320, 330.

First and second ablation pads 222, 232 then respectively may be provided on first and second arms 220, 230 (step 750). For example, pads 222, 232 suitably may be coupled to first and second arms via thermoplastic bonding, adhesives, stitching, or the like. As noted above, ablation pads 222, 232 may have any suitable construction for use in emitting the desired amount and type of ablation energy. For example, pads 222, 232 may be conductive, e.g., metallic, electrodes, configured to emit RF energy generated by an ablation energy generator and coupled thereto by conductors 323, 333. Or, for example, pads 222, 232 may be ultrasonic transducers, such as piezoelectric elements, configured to emit ultrasonic waves of a desired frequency and intensity responsive to control signals generated by a suitable generator and coupled thereto by conductors 323, 333. Or, for example, pads 222, 232 may include microwave antennas configured to emit microwave energy responsive to control signals generated by a suitable generator and coupled thereto by conductors 323, 333.

First and second conductors 323, 333, respectively connected to first and second ablation pads 222, 232 and respectively disposed within conductor lumens 332, 332, and 340 defined through first and second arms 220, 230 and main section 210 then are provided (step 760). Preferably, first and second conductors 323, 333 pass out of the body and are configured to be coupled to one or more ablation energy sources. Each of conductors 323, 333 may include one, two or more individual conductors suitably configured to provide ablation energy and/or control signals to ablation pads 222, 232, and may include thermal and/or electrical insulation as appropriate. Conductor lumens 322, 332, and 340 may be defined during step 720, e.g., at about the same time as guidewire lumens 320, 330.

After the various components of flexible catheter 200 are formed, the catheter may be disposed within a retractable delivery sheath (step 770). For example, as illustrated in FIG. 8A, first and second arms 220, 230 may be folded so as to be adjacent to one another, and main section 210 and folded arms 220, 230 of flexible catheter 200 may be inserted into delivery sheath 800. Proximal end 811 of sheath 800 may be disposed outside of the patient's body, and distal end 812 is configured to be percutaneously deployed within abdominal aorta 130 (not illustrated in FIG. 8A). For example, sheath 800 suitably may have a diameter of 9 French or less, although smaller and larger diameters also suitably may be used. Preferably, the distal ends of first and second arms 220, 230 are disposed at a point proximal of distal end 812 of sheath 800. As illustrated in FIG. 8B, delivery sheath 800 may be retracted proximally (in the −y direction) relative to flexible catheter 200, which exposes first and second arms 220, 230 that then spread apart from one another to respectively be disposed into renal artery 120 and aorta 130 (not illustrated in FIG. 8B). It should be appreciated that the particular angles that first and second arms 220, 230 then obtain relative to one another and to main section 210 may be defined by the trajectories of guidewires 223, 233 passing therethrough (guidewires not illustrated in FIGS. 8A-8B).

It also should be appreciated that the steps of method 700 may be performed in any suitable order relative to one another. For example, steps 710 and 720 may be performed simultaneously with one another such that lumens 320, 330 are formed at the same time as first arm 220, second arm 230, and main section 210. Or, for example, steps 730 and 740 may be performed in the opposite order relative to one another, so that pullwires 351, 371 are in place before joints 213, 214 are formed. Likewise, steps 750 and 760 may be performed in the opposite order relative to one another, so that conductors 323, 333 are in place before first and second ablation pads 222, 223 are provided on first and second arms 220, 230. Further, steps 750 and/or 760 may be performed before steps 730 and/or 740, so that ablation pads 222, 223 and/or conductors 323, 333 are provided before joints 213, 214 and/or pullwires 351, 371. Additionally, conductors 323, 333 respectively may be embedded directly within first arm 220, second arm 230, and main section 210 at the time the first and second arms and main section are prepared (e.g., during step 710) rather than during a separate, subsequent step. Any suitable order of preparing the various components of flexible catheter 200 may be used.

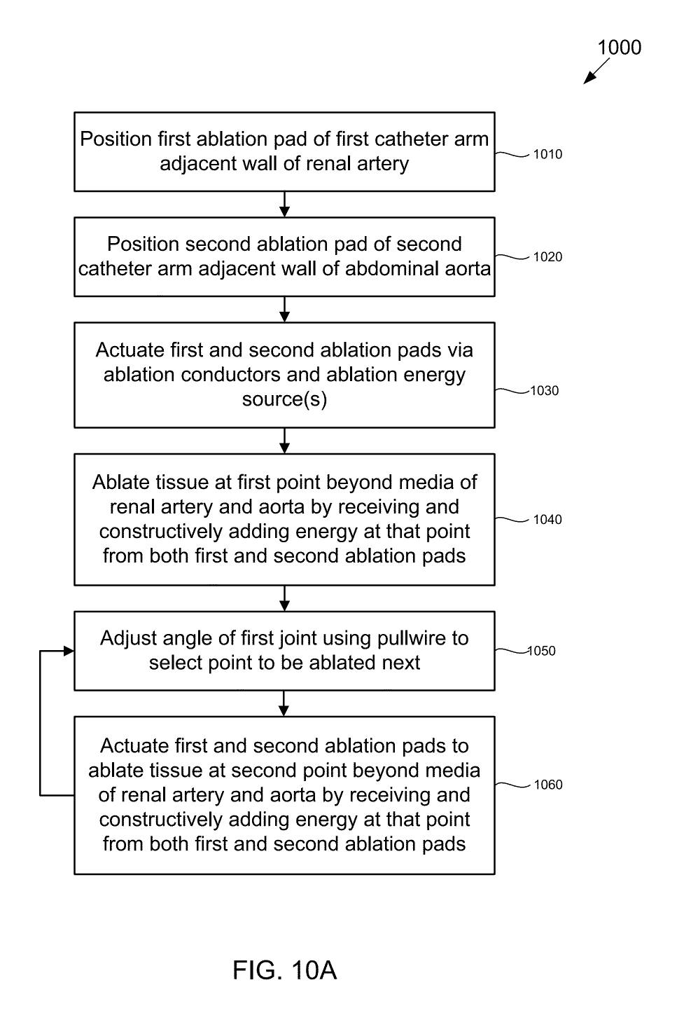

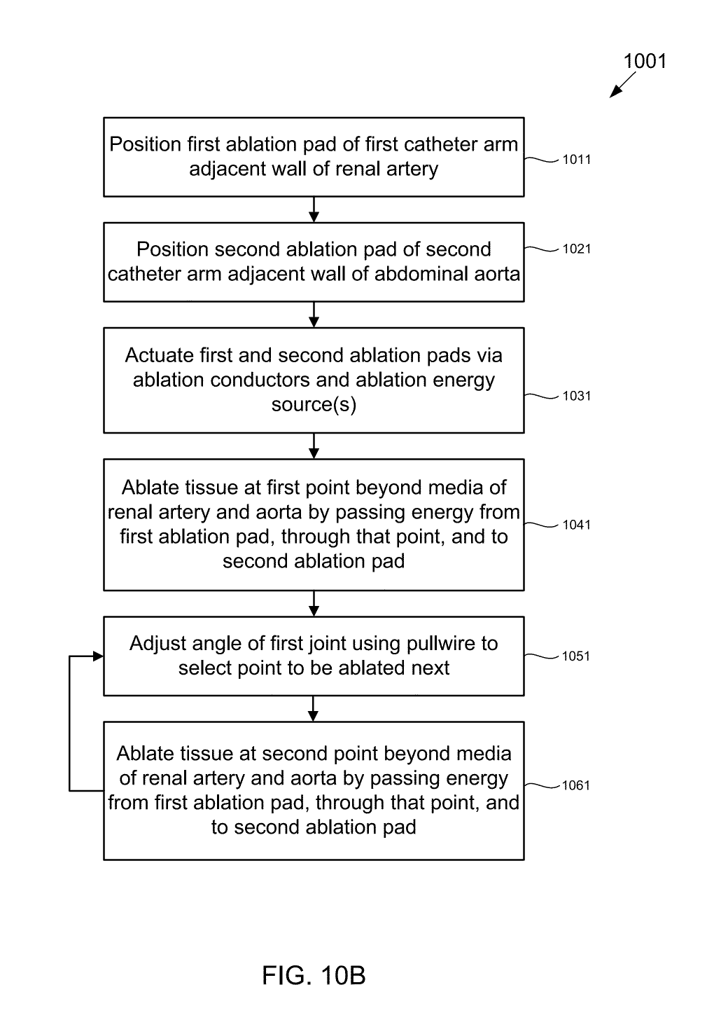

FIG. 9A illustrates steps in an exemplary method 900 of deploying flexible catheter 200 and sheath 800 within a patient's body in preparation for a renal denervation procedure (described further below with reference to FIGS. 10A-10B). Method 900 includes placing aorta guidewire 233 into abdominal aorta 130 (step 910), and placing renal guidewire 223 into renal artery 120 (step 920).

Flexible catheter 200, disposed in delivery sheath 800, is then passed over aorta and renal guidewires 233, 223 (step 930). Specifically, the aorta and renal guidewires 233, 223 are respectively disposed within lumens 320 and 330 that are defined through first arm 220, second arm 230, and main section 210 as illustrated in FIGS. 3A-3I. It should be appreciated that flexible catheter 200 need not necessarily be disposed within delivery sheath 800 before deploying the sheath in aorta 130, and that instead the sheath first may be deployed in the aorta and flexible catheter 200 subsequently advanced through the sheath and into the aorta over guidewires 233, 223.

Regardless of the particular order in which they are deployed in aorta 130, sheath 800 and catheter 200 preferably are positioned so that their respective distal ends are positioned adjacent branchpoint 170 between renal artery 120 and aorta 130, based on imaging and/or tactile resistance (step 940). To facilitate imaging sheath 800 and catheter 200, one or both of these components suitably may include one or more radiopaque markers that may be viewed fluoroscopically by the physician deploying the sheath and catheter. However, such imaging may not be necessary because tactile feedback may be sufficient for the physician to identify when the distal ends of sheath 800 and catheter 200 are positioned adjacent branchpoint 170. Specifically, because renal guidewire 223 has been placed in renal artery 120, and aorta guidewire 233 has been placed in aorta 130, the trajectories of these guidewires are parallel to one another and to aorta 130 proximal branchpoint 170, but diverge from one another at branchpoint 170 as the guidewires travel along their respective blood vessels. As such, sheath 800 and catheter 200 readily may be passed over guidewires 223, 233 at points proximal of branchpoint 170, but may physically resist being advanced beyond branchpoint 170 because, when disposed within sheath 800, catheter 200 cannot simultaneously advance along both of the diverging trajectories of renal guidewire 223 and aorta guidewire 233. The physician deploying flexible catheter 200 and sheath 800 may manually detect such resistance and reasonably conclude that their distal ends are disposed adjacent branchpoint 170. The positions of flexible catheter 200 and sheath 800, if detected based on tactile resistance, optionally also may be confirmed using imaging.

Delivery sheath 800 then may be refracted proximally to expose first and second arms 220, 230 of flexible catheter 200 (step 950). As described above with reference to FIG. 8B, such retraction allows arms 220, 230 to obtain angles relative to each other and to main section 210 that may be defined by the trajectories of guidewires 223, 233 passing therethrough. During step 950 of method 900, arms 220, 230 are entirely proximal of branchpoint 170, in a region in which guidewires 223, 233 are substantially parallel to one another and to aorta 130. As such, arms 220, 230 similarly may be parallel to one another and to aorta 130 during step 950. Delivery sheath 800 may be retracted by a distance sufficient to expose joints 213, 214, so that the joints suitably may be articulated.

For example, angle β of second joint 214 optionally may be adjusted to compensate for the patient's particular anatomy (step 960). Such adjustment may compensate for the particular angle φ/ψ between renal artery 120 and aorta 130, as described above with reference to FIGS. 4A-4B. Depending on the patient's particular anatomy, and the configuration of flexible catheter 200, such adjustment may not be performed.

Flexible catheter 200 then is advanced distally, which advances first arm 220 along renal guidewire 223 into renal artery 120, and advances second arm 230 along aorta guidewire further into aorta 130, until bifurcation 270 between first arm 220 and second arm 230 engages branchpoint 170 between renal artery 120 and aorta 130 (step 970). When bifurcation 270 engages branchpoint 170, flexible catheter 200 may present strong resistance to being advanced any further. Such resistance may be manually detectable by the physician and may allow the physician to confirm that flexible catheter 200 is properly placed and ready to be used to perform renal denervation. The positions of first and second arms 220, 230 optionally also may be confirmed using imaging.

FIG. 9B illustrates steps in an exemplary alternative method 901 of deploying flexible catheter 200 and sheath 800 within a patient's body in preparation for a renal denervation procedure (described further below with reference to FIGS. 10A-10B). Alternative method 901 includes placing renal guidewire 223 into renal artery 120 (step 911). However, in this alternative method, aorta guidewire 233 is not provided and not used.

Flexible catheter 200, disposed in delivery sheath 800, is then passed over renal guidewire 223 (step 921). Specifically, renal guidewire 223 is disposed within lumen 320 defined through first arm 220 and main section 210 as illustrated in FIGS. 3A-3C and 3G-3I. It should be appreciated that flexible catheter 200 need not necessarily be disposed within delivery sheath 800 before deploying the sheath in aorta 130, and that instead the sheath first may be deployed in the aorta and flexible catheter 200 subsequently advanced through the sheath and into the aorta over guidewire 223.

Regardless of the particular order in which they are deployed in aorta 130, sheath 800 and catheter 200 preferably are positioned so that their respective distal ends are positioned adjacent branchpoint 170 between renal artery 120 and aorta 130, based on imaging and/or tactile resistance (step 931). To facilitate imaging sheath 800 and catheter 200, one or both of these components suitably may include one or more radiopaque markers that may be viewed fluoroscopically by the physician deploying the sheath and catheter. However, such imaging may not be necessary because tactile feedback may be sufficient for the physician to identify when the distal ends of sheath 800 and catheter 200 are positioned adjacent branchpoint 170. Specifically, because renal guidewire 223 has been placed in renal artery 120, the trajectory of this guidewire is parallel to aorta 130 proximal branchpoint 170, and then bends through angle φ/ψ at branchpoint 170 as the guidewire travels along renal artery 120. As such, sheath 800 and catheter 200 readily may be passed over guidewire 223 at points proximal of branchpoint 170, but may physically resist being advanced beyond branchpoint 170 because, when disposed within sheath 800, catheter 200 is relatively stiff and has a diameter that may be relatively large compared to renal artery 130, and thus may resist bending through angle φ/ψ to follow renal guidewire 223. The physician deploying flexible catheter 200 and sheath 800 may manually detect such resistance and reasonably conclude that their distal ends are disposed adjacent branchpoint 170. The positions of flexible catheter 200 and sheath 800, if detected based on tactile resistance, optionally also may be confirmed using imaging.

Delivery sheath 800 then may be refracted proximally to expose first and second arms 220, 230 of flexible catheter 200 (step 941). As described above with reference to FIG. 8B, such retraction allows arms 220, 230 to obtain angles relative to each other and to main section 210 that may be defined by the trajectories of guidewires 223, 233 passing therethrough. Delivery sheath 800 may be retracted by a distance sufficient to expose joints 213, 214, so that the joints suitably may be articulated. During step 950 of method 900, arms 220, 230 are entirely proximal of branchpoint 170, in a region in which guidewire 223 is substantially parallel to aorta 130, and in which the angle of arm 230 is unconstrained by a guidewire. As such, arm 220 may be parallel to aorta 130 during step 941, while arm 230 may project relative to arm 220 at a nominal angle that optionally is defined during step 710 of method 700 described above with reference to FIG. 7.

Angle β of second joint 214 optionally then may be adjusted to compensate for the patient's particular anatomy (step 951). Such adjustment may compensate for the particular angle φ/ψ between renal artery 120 and aorta 130, as described above with reference to FIGS. 4A-4B. Depending on the patient's particular anatomy, and the configuration of flexible catheter 200, such adjustment may not be performed.

Flexible catheter 200 then is advanced distally, which advances first arm 220 along renal guidewire 223 into renal artery 120, and advances second arm 230 along further into aorta 130, until bifurcation 270 between first arm 220 and second arm 230 engages branchpoint 170 between renal artery 120 and aorta 130 (step 961). Although second arm 230 is not advanced along a corresponding guidewire, the relative motion of first arm 220 as it enters renal artery 120 may cause second arm 230 naturally to move into aorta 130. When bifurcation 270 engages branchpoint 170, flexible catheter 200 may present strong resistance to being advanced any further. Such resistance may be manually detectable by the physician and may allow the physician to confirm that flexible catheter 200 is properly placed and ready to be used to perform renal denervation. The positions of first and second arms 220, 230 optionally also may be confirmed using imaging.

FIG. 10A illustrates steps in an exemplary method 1000 of performing renal denervation using flexible catheter 200, which may have been deployed using method 900 described above with reference to FIG. 9A or method 901 described above with reference to FIG. 9B.

Method 1000 includes positioning first ablation pad 222 of first catheter arm 220 adjacent the wall of renal artery 120 (step 1010), as well as positioning second ablation pad 232 of second catheter arm 230 adjacent the wall of aorta 130 (step 1020). As illustrated in FIG. 2B, when flexible catheter 200 is properly deployed, first ablation pad 222 contacts the wall of renal artery 120 and second ablation pad 232 contacts the wall of aorta 130. As such, steps 1010 and 1020 of method 1000 naturally may be performed during deployment of flexible catheter 200 during method 900 or 901 respectively described above with reference to FIGS. 9A and 9B.

Then, renal denervation is performed at a first convergence point 241 in the patient's tissue by actuating first and second ablation pads 222, 232 via ablation conductors 323, 333 and ablation energy source(s) (step 1030). Such actuation of first and second ablation pads 222, 232 ablates tissue at first convergence point 241, which lies beyond the media of renal artery 120 and aorta 130, by constructively adding energy at that point from both the first and second ablation pads. Because point 241 is relatively likely to have one or more renal sympathetic nerves 150 passing therethrough, and because ablating that point in such a manner reduces the relative heating of the intima and media of renal artery 120 and aorta 130, renal denervation at point 241 may be achieved.

So as to increase the likelihood of sufficiently damaging renal sympathetic nerves 150 to treat or ameliorate the patient's hypertension, a second convergence point in the patient's tissue may be selected first by adjusting angle γ of first joint 213 using pullwire(s) 371, e.g., as described above with reference to FIGS. 5A-6C (step 1050 of FIG. 10A). First and second ablation pads 222, 232 then may be actuated again to ablate the tissue at the second convergence point, which lies beyond the media of renal artery 120 and aorta 130, by constructively adding energy at that point from both the first and second ablation pads (step 1061).

As noted further above with reference to FIG. 2B, in alternative embodiments first and second ablation pads 222, 232 may be used in a bipolar arrangement in which ablation energy is transmitted from pad 222 to pad 232, or vice versa, that crosses through a desired point 241 to be ablated. FIG. 10B illustrates an alternative method 1001 adapted to perform renal denervation using such a bipolar arrangement.

Like method 1000, method 1001 illustrated in FIG. 10B includes positioning first ablation pad 222 of first catheter arm 220 adjacent the wall of renal artery 120 (step 1011), as well as positioning second ablation pad 232 of second catheter arm 230 adjacent the wall of aorta 130 (step 1021). As illustrated in FIG. 2B, when flexible catheter 200 is properly deployed, first ablation pad 222 contacts the wall of renal artery 120 and second ablation pad 232 contacts the wall of aorta 130. As such, steps 1011 and 1021 of method 1001 naturally may be performed during deployment of flexible catheter 200 during method 900 or 901 respectively described above with reference to FIGS. 9A and 9B.

Then, renal denervation is performed at a first convergence point 241 in the patient's tissue by actuating first and second ablation pads 222, 232 via ablation conductors 323, 333 and ablation energy source(s) (step 1031). Such actuation of first and second ablation pads 222, 232 ablates tissue at first convergence point 241, which lies beyond the media of renal artery 120 and aorta 130, by passing energy from first ablation pad 222, through that point, to second ablation pad 232 (step 1041). Because point 241 is relatively likely to have one or more renal sympathetic nerves 150 passing therethrough, and because ablating that point in such a manner reduces the relative heating of the intima and media of renal artery 120 and aorta 130, renal denervation at point 241 may be achieved.

So as to increase the likelihood of sufficiently damaging renal sympathetic nerves 150 to treat or ameliorate the patient's hypertension, a second convergence point in the patient's tissue may be selected first by adjusting angle γ of first joint 213 using pullwire(s) 371, e.g., as described above with reference to FIGS. 5A-6C (step 1051 of FIG. 10B). First and second ablation pads 222, 232 then may be actuated again to ablate the tissue at the second convergence point, which lies beyond the media of renal artery 120 and aorta 130, by constructively adding energy at that point from both the first and second ablation pads (step 1061).

As noted further above with reference to FIG. 2B, in alternative embodiments first and second ablation pads 222, 232 may be used in a bipolar arrangement in which ablation energy is transmitted from pad 222 to pad 232, or vice versa, that crosses through a desired point 241 to be ablated. FIG. 10B illustrates an alternative method 1001 adapted to perform renal denervation using such a bipolar arrangement.

Note that flexible catheter 200 suitably may be adapted to facilitate direction of ablation energy from first and second ablation pads 222, 232 towards desired point 241 illustrated in FIG. 2B and/or through a plurality of desired points 520 illustrated in FIG. 5D. For example, first and/or second ablation pads 222, 232 may be coupled to suitable control elements (such as pullwire(s)) and may be configured so as to be pivotable in one or two directions responsive to actuation of the control elements. Pivoting first and second ablation pads 222, 232 via such control elements may allow more precise selection of the point(s) to be ablated)

While various illustrative embodiments of the invention are described above, it will be apparent to one skilled in the art that various changes and modifications may be made therein without departing from the invention. For example, although the systems described herein are configured to be compatible with a wide variety of potential patient anatomies, it should be noted that the systems may be modified for use with a more limited range of potential anatomies, or even with a single, particular anatomy, for example by appropriately selecting the diameters and lengths of the main section and the first and second arms, and the angles α, β, and γ described above. In such embodiments, the inclusion of pullwires or other control components suitably may be reduced because the need to adapt the device in situ to the patient's particular anatomy may be reduced. Additionally, it should be recognized that the systems and methods provided herein suitably may be adapted to perform ablation procedures at locations other than at the branchpoint between the renal artery and the abdominal aorta. Indeed, the systems and methods suitably may be adapted for use in ablation procedures to be performed at branchpoints between any two body lumens, including other blood vessels, as well as lumens in the urinary tract, the gastrointestinal tract, the reproductive system, and the like. The appended claims are intended to cover all such changes and modifications that fall within the true spirit and scope of the invention.

What is claimed:

1. A system for performing renal denervation in a patient having an aorta and a renal artery and a branchpoint therebetween, the system comprising:
a flexible catheter comprising a main section, first and second arms, and a bifurcation between the first and second arms,
the main section having a proximal end and a distal end, the distal end configured to be disposed in the aorta,
the first arm being coupled to the distal end of the main section and configured to be disposed in the renal artery,
the second arm being coupled to the distal end of the main section and configured to be disposed in the aorta,
the bifurcation between the first and second arms being configured to engage the branchpoint between the aorta and the renal artery.

2. The system of claim 1, further comprising a first ablation pad coupled to the first arm and a second ablation pad coupled to the second arm such that when the bifurcation between the first and second arms engages the branchpoint between the aorta and the renal artery, the first ablation pad engages a wall of the renal artery, and the second ablation pad engages a wall of the aorta.

3. The system of claim 2, further comprising first and second ablation energy conductors respectively coupled to the first and second ablation pads, the first and second ablation energy conductors each passing through the main section of the flexible catheter and each being configured to be coupled to an ablation energy source.

4. The system of claim 3, wherein the first and second ablation pads each are configured to transmit ablation energy from the ablation energy source to a first convergence point beyond the wall of the renal artery and beyond the wall of the aorta, the ablation energy from the first ablation pad and the ablation energy from the second ablation pad constructively adding at the first convergence point, the constructively added energies being sufficient to ablate tissue at the first convergence point.

5. The system of claim 4, further comprising a first joint along the main section of the flexible catheter, the first joint being articulable in a first plane.

6. The system of claim 5, further comprising a pullwire configured to adjust an angle of the first joint in the first plane so as to configure the first and second ablation pads to transmit ablation energy from the ablation energy source to a second convergence point beyond the wall of the renal artery and beyond the wall of the aorta, the ablation energy from the first ablation pad and the ablation energy from the second ablation pad constructively adding at the second convergence point, the constructively added energies being sufficient to ablate tissue at the second convergence point.

7. The system of claim 5, further comprising a second joint along the main section of the flexible catheter, the second joint being articulable in a second plane that lies substantially orthogonal to the first plane.

8. The system of claim 7, the first and second joints each comprising a shape memory material.

9. The system of claim 3, wherein the first ablation pad is configured to transmit ablation energy from the ablation energy source to the second ablation pad, the ablation energy crossing through a point beyond the wall of the renal artery and beyond the wall of the aorta, the ablation energy being sufficient at the point to ablate tissue at the point.

10. The system of claim 9, further comprising a first joint along the main section of the flexible catheter, the first joint being articulable in a first plane.

11. The system of claim 10, further comprising a pullwire configured to adjust an angle of the first joint in the first plane so as to configure the first ablation pad to transmit ablation energy from the ablation energy source to the second ablation pad through a second convergence point beyond the wall of the renal artery and beyond the wall of the aorta, the ablation energy from the first ablation pad being sufficient to ablate tissue at the second convergence point.

12. The system of claim 2, wherein the first and second ablation pads are configured to emit unipolar radiofrequency (RF) energy, bipolar RF energy, ultrasonic waves, microwave energy, irreversible electroporation, or ionizing radiation.

13. The system of claim 2, further comprising a sensor configured to sense a parameter and a programmable controller configured to receive the sensed parameter from the sensor, the programmable controller further configured to direct the first and second ablation pads to emit energy based on the sensed parameter.

14. The system of claim 1, further comprising a sheath configured to be disposed within the aorta, the flexible catheter being disposed within the sheath, the sheath being retractable relative to the flexible catheter.

15. The system of claim 14, further comprising:
a guidewire configured to be disposed within the renal artery; and
a guidewire lumen defined through the flexible catheter and configured to receive the guidewire,
the flexible catheter disposed in the sheath being guidable to a point adjacent the renal artery by pushing the flexible catheter over the guidewire,
the flexible catheter being configured such that retraction of the sheath at the point exposes the first and second arms and the bifurcation therebetween,
the flexible catheter being configured such that advancement of the flexible catheter after exposing the first and second arms causes the bifurcation between the first and second arms to engage the branchpoint between the renal artery and the aorta.

16. The system of claim 14, further comprising a pullwire and a joint along the main section of the flexible catheter, the joint being articulable in a first plane, an angle of the joint being selectable by retracting the pullwire so as to apply additional force in a direction normal to the branchpoint between the renal artery and the aorta.

17. A method for performing renal denervation in a patient having an aorta and a renal artery and a branchpoint therebetween, the method comprising:
providing a flexible catheter comprising a main section, first and second arms, and a bifurcation between the first and second arms, the main section having a proximal end and a distal end, the distal end configured to be disposed in the aorta, the first and second arms each being coupled to the distal end of the main section;
disposing the first arm in the renal artery;
disposing the second arm in the aorta; and
engaging the branchpoint between the aorta and the renal artery with the bifurcation between the first and second arms.

18. The method of claim 17, wherein the flexible catheter further comprises a first ablation pad coupled to the first arm and a second ablation pad coupled to the second arm, the method further comprising engaging the bifurcation between the first and second arms with the branchpoint between the aorta and the renal artery such that the first ablation pad engages a wall of the renal artery and the second ablation pad engages a wall of the aorta.

19. The method of claim 18, wherein the flexible catheter further comprises first and second ablation energy conductors respectively coupled to the first and second ablation pads, the first and second ablation energy conductors each passing through the main section of the flexible catheter, the method further comprising coupling the first and second ablation energy conductors to an ablation energy source.

20. The method of claim 19, further comprising transmitting ablation energy from the ablation energy source from the first and second ablation pads to a first convergence point beyond the wall of the renal artery and beyond the wall of the aorta, the ablation energy from the first ablation pad and the ablation energy from the second ablation pad constructively adding at the first convergence point, the constructively added energies being sufficient to ablate tissue at the first convergence point.

21. The method of claim 20, wherein the flexible catheter further comprises a first joint along the main section of the flexible catheter, the method further comprising articulating the first joint in a first plane.

22. The method of claim 21, further comprising adjust an angle of the first joint in the first plane via a pullwire such that the first and second ablation pads each transmit ablation energy from the ablation energy source to a second convergence point beyond the wall of the renal artery and beyond the wall of the aorta, the ablation energy from the first ablation pad and the ablation energy from the second ablation pad constructively adding at the second convergence point, the constructively added energies being sufficient to ablate tissue at the second convergence point.

23. The method of claim 21, wherein the flexible catheter further comprises a second joint along the main section of the flexible catheter, the method further comprising articulating the second joint in a second plane that lies substantially orthogonal to the first plane.

24. The method of claim 23, the first and second joints each comprising a shape memory material.

25. The method of claim 19, further comprising transmitting ablation energy from the ablation energy source from the first ablation pad to the second ablation pad, the ablation energy crossing through a point beyond the wall of the renal artery and beyond the wall of the aorta, the ablation energy being sufficient at the point to ablate tissue at the point.

26. The method of claim 25, wherein the flexible catheter further comprises a first joint along the main section of the flexible catheter, the method further comprising articulating the first joint in a first plane.

27. The method of claim 26, further comprising adjusting an angle of the first joint in the first plane via a pullwire such that the first ablation pad transmits ablation energy from the ablation energy source to the second ablation pad through a second convergence point beyond the wall of the renal artery and beyond the wall of the aorta, the ablation energy from the first ablation pad being sufficient to ablate tissue at the second convergence point.

28. The method of claim 17, further comprising:
disposing the flexible catheter within a sheath, the sheath being retractable relative to the flexible catheter; and
disposing the flexible catheter within the sheath within the aorta.

29. The method of claim 28, further comprising:
disposing a guidewire within the renal artery;
receiving the guidewire within a guidewire lumen defined through the flexible catheter;
guiding the flexible catheter disposed in the sheath to a point adjacent the renal artery by pushing the flexible catheter over the guidewire;
retracting the sheath at the point to expose the first and second arms and the bifurcation therebetween;
advancing the flexible catheter after exposing the first and second arms to engage the bifurcation between the first and second arms with the branchpoint between the renal artery and the aorta.

30. The method of claim 29, wherein the flexible catheter further comprises a pullwire and a joint along the main section of the flexible catheter, the method further comprising articulating the joint in a first plane and selecting an angle of the joint by retracting the pullwire so as to apply additional force in a direction normal to the branchpoint between the renal artery and the aorta.
